(19) United States
(12) Patent Application Publication
(10) Pub. No.: US 2025/0312559 A1
(43) Pub. Date: Oct. 9, 2025

(54) Targeted Brain Rehabilitation System and Therapeutic Method for Treating Phantom Limb Syndrome

(71) Applicant: XR Technologies, LLC, Alpharetta, GA (US)

(72) Inventors: Ryan Serbin, Charlotte, NC (US); James Tyler Frix, Charlotte, NC (US); Raymond Glenn Gaston, Charlotte, NC (US); Bryan Loeffler, Charlotte, NC (US)

(21) Appl. No.: 19/043,878

(22) Filed: Feb. 3, 2025

Related U.S. Application Data
(60) Provisional application No. 63/557,857, filed on Feb. 26, 2024.
(60) Provisional application No. 63/725,506, filed on Nov. 26, 2024.

(51) Int. Cl. A61M 21/02; A61M 21/00; G06F 3/01

(52) U.S. Cl. CPC A61M 21/02; G06F 3/015; A61M 2021/0011; A61M 2021/005; A61M 2205/507; A61M 2230/08

(57) ABSTRACT

Disclosed is a system and method for administering targeted brain rehabilitation therapies in an immersive multimedia environment so as to reduce phantom limb pain and counteract cortical remapping associated with limb amputation in a patient user of the system and method. Embodiments of the solution leverage immersive extended reality environments, such as augmented (AR) and virtual (VR) reality, which may be combined with autonomous limb tracking sensors, myoelectric equipment, custom-tailored projections of a user's amputated limb(s), and a progressive therapeutic regimen based on Graded Motor Imagery (GMI) and mirror therapy, among other features and aspects.

### CROSS-REFERENCE TO RELATED APPLICATIONS

Priority under 35 U.S.C. § 119(e) is claimed to the U.S. provisional application entitled “ENHANCED VIRTUAL REALITY SYSTEM FOR PHANTOM LIMB PAIN REHABILITATION AND THERAPY,” filed on Feb. 26, 2024, and assigned application Ser. No. 63/557,857, the entire contents of which are hereby incorporated by reference. Priority under 35 U.S.C. § 119(e) is also claimed to the U.S. provisional application entitled “TARGETED BRAIN REHABILITATION: DEVELOPMENT, FEASABILITY, AND USABILITY OF A NOVEL VIRTUAL REALITY SYSTEM FOR PHANTOM LIMB PAIN MANAGEMENT AND AMPUTEE REHABILITATION,” filed on Nov. 26, 2024, and assigned application Ser. No. 63/725,506, the entire contents of which are hereby incorporated by reference.

### BACKGROUND

Phantom Limb Pain (PLP) is a condition in which amputees experience pain in a limb no longer present, affecting approximately 80% of amputees. Patients with a PLP condition tend to describe the sensations associated with PLP from a slight tingling feeling to the feeling of being stabbed or being burned with a hot iron. They also describe mechanical pain such as that derived from the absent limb being contorted into painful positions, such as a clinched fist with fingernails being buried into the palm or an arm twisted behind their back. Because the brain of a PLP patient renders nerve impulses from the neurons formerly connected to a lost limb as pain, physicians often use opioids to treat PLP conditions, with a patient's increased reliance on opioids over time to manage the condition as an unfortunate result. Interestingly, however, opioid therapy is only effective against PLP for about fifty percent of patients. Even so, an increased reliance over time on opioids still often manifests with PLP patients as they use the opioids to manage the stress and anxiety that comes with the intangible and invisible nature of living with a PLP condition.

At its best, treating PLP conditions with opioids or other pharmacologic agents is little more than putting the proverbial “band aid on the symptom” because the regimen does not treat the cortical remapping that takes place post-amputation, whereby cortical regions linked to the lost limb are crowded out by surrounding sensory regions, contributing to the development and persistence of PLP. As such, opioids only serve to dampen PLP and do not do anything to treat its actual underlying cause. As we understand it, PLP arises from both central nervous system (CNS) and peripheral nervous system (PNS) drivers. Peripherally, neuroma formation can lead to radiating phantom sensations that are quite painful. Centrally, cortical remapping leads to PLP as the areas in the sensorimotor cortex responsible for the phantom limb are crowded out overtime by adjacent sensory regions causing the development of PLP and increasing the difficulty of treatment/therapy success. For these reasons, it is evident that narcotic therapy was never going to be a successful long term treatment option in amputee patients. The unimpressive efficacy, non-curative approach and significant risk of opioid reliance, make it clear that other non-opioid treatment systems are desirable. For PNS drivers of phantom limb pain, targeted muscle reinnervation (TMR) and regenerative peripheral nerve interface have arisen as good surgical options to treat and prevent PLP due to neuroma formation. However, the CNS drivers of phantom limb pain have proven more difficult to treat. Current alternatives in the art include meditation, cognitive behavioral therapy, mirror therapy, transcutaneous electrical nerve stimulation, and spinal cord stimulation.

The current art demonstrates that daily use of a prosthesis helps prevent such cortical remapping, reducing associated PLP. As such, integration of active prosthesis use instead of a single-pronged opioid approach is paramount toward satisfactory patient outcomes. Unfortunately, however, getting fit with a prosthesis can take months to years and, with each passing day, the deleterious effects of cortical remapping make eventual prosthesis use more difficult. When amputees do receive their prosthesis they frequently discontinue use before becoming a daily active user due to comfort and function issues with the device that must be corrected, allowing cortical remapping to continue. A more feasibly-adoptable method is needed.

To this end, research in Mirror Therapy (MT) and Graded Motor Imagery (GMI) has shown promise in addressing cortical remapping by stimulating relevant cortical regions; however, these methods as currently practiced are limited to 2-dimensional, in-person use, lacking the structured, progressive, and sensory-immersive therapeutic experience offered by this new system. The current art also lacks a trackable, progressive protocol that patients can use both in a clinical and home environment, limiting patient access and usability which is critical (as of this writing, there are over 2 million amputee patients in the United States served by just 12 specialty clinics, making geographic distance one of the primary barriers to amputee patients receiving the therapy they need).

Therefore, there is a need in the art for a system and method that addresses the shortcomings outlined above. More specifically, there is a need in the art for a Targeted Brain Rehabilitation (TBR) system that uniquely harnesses XR technology with GMI techniques to help patients combat cortical remapping and the loss of left-right discrimination post-amputation in a 3-dimensional, scalable, trackable manner. Moreover, there is a need in the art for a TBR system comprising four treatment prongs delivered via XR methods: laterality recognition, guided meditation, virtual mirror feedback, and guided motor execution.

### BRIEF SUMMARY

The presently disclosed embodiments, as well as features and aspects thereof, are directed towards providing a system and method for reducing phantom limb pain and counteracting cortical remapping associated with limb amputation. Embodiments of the solution leverage immersive extended reality environments, such as augmented (AR) and virtual (VR) reality, which may be combined with autonomous limb tracking sensors, myoelectric equipment, custom-tailored projections of a user's amputated limb(s), and a progressive therapeutic regimen based on Graded Motor Imagery (GMI) and mirror therapy, among other features and aspects. Advantageously, embodiments of the solution may provide for the provision of the therapeutic regimen in either a clinical environment or at home and allow for use as early as the immediate peri-operative setting, thus stopping cortical remapping before it starts.

Components of embodiments are integrated within a dynamic application that allows direct interaction with the patient and their core treatment team (clinicians such as, but not limited to, physician, therapist, prosthatists, etc.) and creates an engaging social/competitive virtual environment. Embodiments of the solution allow the patient access to an immediate cohort of credentialed therapists, built-in therapy assistants, and patient peers to help guide them through their rehabilitation.

Embodiments of the solution, when used by an amputee user soon after amputation, will begin retraining the amputee's remaining stump musculature to operate a myoelectric or other prosthesis, through integration of myoelectric training bands and other devices. Advantageously, immediate use in the perioperative period may provide adjunctive post operative pain relief through both distraction and CNS stimulation. Early adoption of the solution by an amputee user may work to prevent cortical remapping in the user's brain and/or phantom limb pain. For those amputee users already suffering from phantom limb pain and cortical remapping, however, embodiments of the solution will retrain the brain to help reverse the deleterious effects of cortical reorganization with a progressive, complex neuropsychological process and step by step protocol that includes left-right discrimination and other therapeutic phases/goals as will become clearer from further disclosure. Therapeutic algorithms employed by the solution may work to convert amputees into daily active prosthesis wearers by ensuring they are properly trained on a prosthetic before being fitted with the prosthetic.

Moreover, it is an advantage of the solution that it may present a viable, non-pharmacological alternative to narcotic/opioid pain medication for managing recurrent phantom limb pain/discomfort, which is accessible around-the-clock without the adverse side effects seen with pharmacologic or surgical treatment. Moreover, in addition to amputees, it is envisioned that certain embodiments of the solution may be extended to, and employed by, all acutely ill or chronic pain patients, providing a means to escape their immediate reality. The diversion provided by administering therapeutic regimens in a virtual environment may decrease the usage of narcotic pain medication and enhance both psychosocial patient-reported outcomes and hospital metrics (e.g., length of stay, level of acuity, etc.).

Acknowledging the extensive applicability of GMI, embodiments of the solution are thoughtfully engineered to transcend the confines of phantom limb pain management and revolutionize the therapeutic landscape for a wide spectrum of conditions, including neuropathic pain, CRPS, post-operative pain, and post-stroke rehabilitation. In doing so, the solution may address a crucial gap in current therapeutic practices, offering a dynamic, interactive platform for comprehensive pain management and motor-sensory rehabilitation across diverse patient demographics.

An exemplary embodiment of a method for administering Targeted Brain Rehabilitation therapies in an immersive multimedia environment comprises generating an immersive multimedia environment, rendering a first avatar within the immersive multimedia environment (wherein the first avatar is associated with a patient user of an XR subsystem and includes a virtual representation of a lost limb(s) of the patient user), and administering a Targeted Brain Rehabilitative therapy exercise to the patient user via the first avatar (wherein the therapy exercise in the immersive multimedia environment works to address somatosensory and kinesthetic symptoms associated with the patient user's lost limb).

The exemplary embodiment further comprises monitoring myoelectrical signals associated with a remaining musculature of the patient user, identifying myoelectrical signal patterns associated with stimuli presented to the patient user via the immersive multimedia environment, and manipulating the virtual representation of the lost limb in the immersive multimedia environment.

The targeted brain rehabilitative therapy exercise in the exemplary embodiment may be directed to laterality recognition or imagined movements of the lost limb. The exemplary method further provides for the first avatar to include a virtual representation of a remaining, intact limb of the patient user such that the targeted brain rehabilitative therapy exercise is directed to virtual mirror therapy wherein the virtual representation of the lost limb is manipulated to mirror movement and positioning of the virtual representation of the patient user's remaining, intact limb.

The different exemplary embodiments of a TBR system for administering Targeted Brain Rehabilitation therapies may be operable to provide users with a summative score internally by the application or externally through the application by physicians, therapists, prosthetists, etc. These scores may be used to track user-progress, as goals, for research purposes, and/or to adapt the Targeted Brain Rehabilitation therapy as the user progresses and unlock sections or difficulty levels within the system. This also provides the basis for adaptive neural networks or other forms of artificial intelligence to act as an intra-application personalized therapist to direct user-specific therapy.

### BRIEF DESCRIPTION OF THE DRAWINGS

In the drawings, like reference numerals refer to like parts throughout the various views unless otherwise indicated. For reference numerals with letter character designations such as “102A” or “102B”, the letter character designations may differentiate two like parts or elements present in the various figures. Letter character designations for reference numerals may be omitted when it is intended that a reference numeral to encompass all parts having the same reference numeral in all figures.

FIG. 1 depicts a high-level, functional block diagram of a Targeted Brain Rehabilitation platform (“TBR platform”) according to the solution;

FIG. 2 depicts a functional block diagram of an exemplary user's XR subsystem that may be employed within a TBR platform;

FIG. 3 depicts a functional block diagram of an exemplary TBR module within a TBR platform;

FIG. 4 illustrates an XR environment created by a TBR platform for hosting a TBR session and administering a TBR therapy;

FIG. 5 is a flowchart illustrating an exemplary method for provisioning an exemplary TBR platform for use by a patient user;

FIG. 6 is a flowchart illustrating an exemplary method for providing TBR therapy sessions to a patient user of a TBR platform according to the solution;

FIG. 7 is a flowchart illustrating an exemplary method for providing TBR therapy in the form of a laterality recognition therapy to a patient user of a TBR platform according to the solution;

FIG. 8 is a flowchart illustrating an exemplary method for providing TBR therapy in the form of an imagined movements (“IM”) therapy to a patient user of a TBR platform according to the solution;

FIG. 9 is a flowchart illustrating an exemplary method for providing TBR therapy in the form of a virtual mirror (“VM”) therapy to a patient user of a TBR platform according to the solution;

FIG. 10 is a flowchart illustrating an exemplary method for myoelectric prosthesis training of a patient user of a TBR platform according to the solution;

FIG. 11 is a schematic diagram illustrating an exemplary software architecture of the TBR platform of FIG. 1 for providing TBR therapy sessions via an immersive multimedia workload;

FIG. 12 is a diagram of the stepwise progression of the four phases of TBR therapy that may be implemented in a virtual environment by and through embodiments of the solution;

FIG. 13 illustrates an amputee user of an embodiment of the solution that leverages an XR headset along with both embedded and external sensors to scan the user's intact limb and mirror a copy onto the user's avatar in the XR environment to represent the user's amputated limb;

FIG. 14 illustrates an amputee user of an embodiment of the solution that leverages an XR headset along with both internal and external sensors (such as a myoelectric band and EEG activity sensors) to map the desired location and movement of the user's amputated/phantom limb and project an anatomically appropriate copy onto the user's avatar in the XR environment to represent the user's amputated limb;

FIG. 15 illustrates an amputee user of an embodiment of the solution that leverages an XR headset along with both internal and external sensors (such as a myoelectric band and EEG activity sensors) to map the desired location and movement of the user's amputated/phantom limb and project a virtual representation of a correctly positioned given/selected prosthetic onto the user's avatar in the XR environment;

FIG. 16 illustrates an exemplary embodiment of a Gaze Directed Interaction (GDI) functionality that may be included in the user interface of certain embodiments of the solution;

FIG. 17 is a further illustration of the GDI functionality shown and described by FIG. 16;

FIG. 18A illustrates an exemplary embodiment of a Random Pose Generator (RPG) functionality that may be included in certain embodiments of the solution, the exemplary embodiment configured to present a hand pose selected from a predefined set of hand poses;

FIG. 18B illustrates an exemplary embodiment of a Random Pose Generator (RPG) functionality that may be included in certain embodiments of the solution, the exemplary embodiment configured to present a randomly generated hand pose; and

FIGS. 19A and 19B are more detailed high-level illustrations of the exemplary architecture for a TBR system shown in FIGS. 1 and 4.

### DETAILED DESCRIPTION

The presently disclosed embodiments, as well as features and aspects thereof, are directed towards a Targeted Brain Rehabilitation system and therapeutic method for treating phantom limb pain syndrome (“TBR system” or “TBR platform”). Embodiments of the TBR platform solution comprise an XR system configured for administering and tracking therapeutic methods by and through a virtual and/or augmented reality. Moreover, certain embodiments of the solution may identify nerves in an amputee's residual musculature, such as a residual limb or chest, and map nerve activity to stimuli presented to the user while employing a therapeutic methodology in a virtual or augmented reality environment.

As will become clear from the figures and explanations provided herein, certain embodiments of the solution for a TBR platform treat phantom limb pain (“PLP”) by administering evidence-based therapies (such as mirror therapy, graded motor imagery, phantom motor execution, etc.) through an extended reality system. By using a four-tiered, customizable treatment approach including laterality recognition, guided meditation, virtual mirror feedback, and guided motor execution, the solution helps to address both central and peripheral contributors to phantom limb pain and reduce, and/or reverse, the cortical remapping that results post-amputation. An advantage of a TBR platform according to the solution is that amputee users who are physically remote from clinicians and/or caregivers may engage in PLP treatment and amputee rehabilitation sessions in a virtual environment remotely accessible by both the patient and the clinician, making available at-home, comprehensive care for patients suffering from PLP. A more detailed explanation of a novel TBR system and its various aspects follows.

The word “exemplary” is used herein to mean “serving as an example, instance, or illustration.” Any aspect described herein as “exemplary” is not necessarily to be construed as exclusive, preferred or advantageous over other aspects.

In this description, the term “Targeted Brain Rehabilitation” (“TBR”) refers to a scalable treatment method administered in an XR environment to provide treatment for a patient user such as, but not limited to, an amputee user suffering from PLP. TBR incorporates a structured, four-phase approach to gradually engage and rewire cortical regions in the brain that are associated with the PLP condition and/or prevent those cortical maladaptions before they set in. The four phases of TBR work to sequentially activate the premotor, supplementary motor, and primary motor cortices of an amputee patient through laterality recognition (distinguishing “left” from “right”), guided meditation, virtual mirror feedback, and guided motor execution. The premotor cortex is activated through planning movements based on external stimuli. The supplementary motor cortex oversees planning/rehearsing complex limb movements from memory and is activated by actively imagining a movement. The primary motor cortex is involved in executing movements through active muscle contractions. Of note, the somatosensory cortex lies directly adjacent to the primary motor cortex and can also be activated through these pathways. More specifically, the laterality recognition phase prompts a user to identify randomly generated, three-dimensional hand postures to engage the user's premotor cortex. Unbeknownst to the user, when they are presented a 3D rendition of a limb the premotor cortex activates as the user unknowingly rotates the limb in their mind based on the external stimulus to reposition it in a more recognizable posture that the user can then identify as left or right. As the premotor cortex is directly adjacent to the motor and supplementary motor cortices, the activation of the former helps to prime the latter for activation (phases 2-4). If the supplementary motor and primary motor cortices are activated before they are primed and ready in a patient with chronic PLP or chronic regional pain syndrome, then activation of these areas can cause severe pain/discomfort. The guided meditation phase engages the user in immersive explicit motor imagery exercises to engage the user's premotor cortex and supplementary motor area. The patient is prompted, through a meditative exercise, to imagine movements of their limbs. This activates the supplementary motor cortex and gets them one step closer to being ready for primary motor cortex activation. As will become clear from subsequent disclosure and figures, the solution may measure, record, and act upon a patient's pain feedback; so, if a patient has increasing pain after phases 1 or 2 then they can be prompted to repeat these phases before proceeding on to later phases. The virtual mirror feedback phase presents the user with bilateral movements of a virtual representation of the user's amputated limb, based on actual bilateral movements controlled by the user's remaining, intact limb, to engage the user's primary motor cortex. As will become clear from subsequent disclosure and figures, the solution may allow for scanning of the user's intact limb to replicate the appearance, size, texture, etc and duplicate that appearance to the 3D avatar of the missing limb in the extended reality environment. Advantageously, doing so helps to make the mirror feedback phase as realistic as possible, ensuring thorough activation of the primary motor cortex. Finally, the guided motor execution phase works to allow independent control of the phantom limb. This phase requires the activation of the premotor, supplementary motor, and active motor cortex. As will become clear from subsequent disclosure and figures, the solution may generate an avatar of the user's missing limb in the 3D mixed reality environment and then take the user, via the avatar, through a series of animated movements. in doing so, the amputee user may be asked to plan and execute these movements with the missing limb, activating both the phantom and the remaining limb musculature.

In this description, the terms “limb,” “extremity,” and “body part” are used broadly to refer to any anatomical limb (arm, leg, etc.), or appendage of a human user of a TBR platform. It is envisioned that embodiments of the solution for a TBR platform may be used for the benefit of an amputee patient user to effectively and efficiently administer and document TBR therapies and related methodologies, without limitation as to the particular limb or appendage, or partial limb or partial appendage, that the user may no longer have. As such, unless stated as specifically limited to a certain limb or appendage of a user (e.g., a left arm, or a right hand), the reader will understand that use of the term “limb” does not limit the scope or applicability of the disclosed solutions. In fact, it is envisioned that embodiments of the solution may be applicable and useful for treating stroke victims, chronic regional pain syndrome (CRPS), brachial plexus and peripheral nerve injuries, as well as those with cortical misrepresentations of existing limb(s) who may not have not suffered in any way from a lost limb.

In this description, “cortical remapping” refers to the process whereby the brain regions responsible for sensory and motor control of a lost limb undergo neuroplastic changes such that their nerve inputs and outputs are lost. Similarly, “cortical encroachment” is the biological process by which nearby cortical regions in a brain crowd out the cortical space previously linked to a lost limb.

In this description, it will be understood that a “user” of a TBR system according to the solution may be any one or more of a clinical patient in need of a TBR-based therapy (such as, but not limited to, an amputee, a stroke victim, chronic regional pain syndrome patient, nerve injury, etc.), a clinician (such as, but not limited to, a therapist, a physician, a physician's assistant, a prosthetists, etc.), an administrator, a caregiver, or any other person interacting with the TBR system, whether by and through use of a headset, a “desktop” computer interface, smartphone/tablet, or the like. It is expected that one of ordinary skill in the art reading this disclosure will understand from context, if not through explicit description, the classification and role of a given exemplary user of an exemplary TBR system embodiment, or aspect thereof, being described.

In this description, the term “application” may include files having executable content, such as: object code, scripts, byte code, markup language files, and patches. In addition, an “application” referred to herein may also include files that are not executable in nature, such as documents that may need to be opened or other data files that need to be accessed.

As used in this description, the terms “component,” “database,” “module,” “system,” “processing component,” “engine,” and the like are intended to refer to a computer-related entity, either hardware, firmware, a combination of hardware and software, software, or software in execution. Notably, and as would be understood by one of ordinary skill in the art, software cannot exist apart from computer memory (i.e., computer readable media) and cannot be executed apart from computer processing components. With the above in mind, a component may be, but is not limited to being, a process running on a processor, a processor, an object, an executable, a thread of execution, a program, and/or a computer. By way of illustration, both an application running on a computing device and the computing device may be a component. One or more components may reside within a process and/or thread of execution, and a component may be localized on one computer (such as an XR headset subsystem) and/or distributed between two or more computers (such as between a cloud-based server and an XR headset). In addition, these components may execute from various computer readable media having various data structures stored thereon. The components may communicate by way of local and/or remote processes such as in accordance with a signal having one or more data packets (e.g., data from one component interacting with another component in a local system, distributed system, and/or across a network such as the Internet with other systems by way of the signal).

In this description, the term “platform” refers to any combination of hardware devices, operating systems, and virtual machines that form an infrastructure upon which software may be executed. A platform may comprise components, modules, databases, etc. Accordingly, the term “TBR platform” refers to any platform configured and operable to implement, administer, and employ TBR methodologies according to the solution in an XR environment. As such, a TBR platform may encompass and leverage an XR platform configured to generate and render an immersive multimedia experience through a virtual and/or augmented reality environment, as will become evident to one of ordinary skill in the art reviewing this disclosure.

In this description, the terms “central processing unit (“CPU”),” “digital signal processor (“DSP”),” “graphical processing unit (“GPU”),” “application specific integrated circuit (“ASIC”), and “chip” are used to refer to exemplary processing components that may be processing a workload according to an immersive multimedia application. As such, the terms “processing component” and “processor” are used interchangeably in this description to refer to any one or more of a CPU, DSP, GPU, ASIC, or a chip or the like. A module may comprise one or more of these processing components. Moreover, a CPU, DSP, GPU, ASIC or a chip may be comprised of one or more distinct processing components generally referred to herein as “core(s).”

In this description, the terms “workload,” “process load,” “process workload,” “immersive multimedia workload,” “XR workload,” “GMI workload,” “TBR workload” and the like are generally directed toward the processing burden, or percentage of processing burden, that a given processing component(s) or module in a given embodiment of the solution may bear to execute, render and provide functionality of a TBR methodology in an XR environment.

In this description, the term “XR” is used as an umbrella term that refers generally to an extended reality environment of any type operable to generate and deliver an immersive multimedia content and, therefore, encompasses a broad spectrum of immersive technologies, including augmented reality (“AR”), virtual reality (“VR”), and mixed reality (“MR”). Whether a given embodiment of the solution leverages a purely VR environment, a purely AR environment, or an MR environment that combines elements of VR and AR, the term XR is used herein to refer to any and all of those different technologies and approaches for merging or replacing real-world experiences with computationally manipulated content. XR is intentionally and primarily used in this description because it is envisioned that the scope of the solution described herein is not limited to any specific type of immersive technology (unless specifically stated or claimed otherwise). One of ordinary skill in the art would understand and acknowledge that an XR system covers the entire spectrum of reality technologies, from fully real to fully virtual environments and everything in between. To this end, an XR system may involve augmenting a user's perception of the real world, immersing a user into an entirely digital realm, or blending elements of both to create interactive mixed reality environments.

In this description, Head Mounted Display (“HMD”), and Heads Up Display (“HUD”), and “headset,” and “glasses” are used interchangeably, unless specifically stated otherwise, to refer to a component of an XR system that is worn by a user so that the user can view and interact with immersive multimedia digital content generated and rendered by the XR platform according to the TBR system. Smart glasses, specifically, are generally associated with AR-type systems, as the glasses are designed to present digital content in an overlay as the user simultaneously views the real-world environment. Smart glasses and headsets come in various forms, ranging from smartphone-compatible devices to standalone headsets with built-in processors. HMDs allow users to see digital content overlaid on their physical environment (such as in an AR-type XR platform) or experience fully immersive virtual worlds (such as in a VR-type XR platform). As would be understood by those with skill in the art, HMDs used by XR platforms often comprise camera subsystems and/or computer vision sensors (infrared, LiDAR, etc.) and screen components to capture the user's real-world environment and project digital content onto the user's field of view that is representative of the real-world environment. HMDs, depending on embodiment, may use one or more camera subsystems to track the user's position and environment, while the screen or display projects digital content in real-time. In AR-type applications, a camera subsystem in an HMD may be used to overlay digital elements onto a real-time video of, or even actual view of, the physical world, creating a blended environment where real and virtual objects seem to the user to coexist. In other XR applications, an HMD fitted with computer vision sensors may enable a more dynamic interaction between physical and virtual elements, allowing users of the XR platform to affect or manipulate digital content directly.

In this description, the term “computer vision sensor” refers to any of a group of sensor types, unless specifically stated or claimed to reference a particular type of sensor, operable to generate output signals representative of a real-world environment within a user's proximity. In this way, computer vision sensors generate a three-dimensional model of the user's environment including, but not necessarily limited to, the user's own hand or limb. Some computer vision sensors, but not all, are image sensors typically associated with camera subsystems. Using the signal output of a computer vision sensor, an XR platform may present to a user a real-time, virtual representation of the user's own hand and/or actual environment. Nonlimiting examples of computer vision sensors that may be leveraged by an XR platform are LiDAR sensors (light detection and ranging), infrared sensors, charge coupled device (CCD) sensors, complimentary metal-oxide semiconductor (CMOS) sensors, etc.

In this description, the term “myoelectric band” refers to a user-fitted sensor or array of sensors configured to engage with a user's residual limb and detect electrical signals associated with intentional muscle movements and involuntary muscle flickers. A myoelectric band may translate the detected muscle movements or flickers into electrical signals useful for a TBR system.

As stated above, embodiments of a TBR platform according to the solution comprise an XR system configured for administering and tracking therapeutic methods by and through a virtual and/or augmented reality. Moreover, certain embodiments of the solution, such as when administering a motor execution exercise, may identify nerves in an amputee's residual limb or chest and map nerve activity to stimuli presented to the user while employing a therapeutic methodology in a virtual or augmented reality environment. Similarly, embodiments may monitor electrical activity associated with electroencephalogram (EEG) analysis, spinal cord activity, etc. in response to stimuli presented to the user in the virtual or augmented reality environment. Such neural activity may be monitored with implantable and/or surface monitors/electrodes, as would be understood by one of ordinary skill in the art.

An XR system configured to implement embodiments of the solution may include, at a general level, various hardware components such as, but not limited to, one or more user headsets, tracking sensors, processors/chips in communication with memory components, cameras, and data input devices (including controllers and/or motion trackers), as would be understood by one of ordinary skill in the art of XR systems. Embodiments of the solution may further include a TBR software platform that is configured to be executed by and through the hardware components of the XR system to create and manage a virtual environment useful for administering one or more TBR therapies. To this end, a TBR software platform may, for example, render graphics, process user and sensor inputs, manage simulations, collect and store progress data and other data, etc.

As would be acknowledged by one of ordinary skill in the art of XR systems, an HMD is the primary device for presenting a virtual environment to a user/wearer and so is the means by which a user of an XR system is visually, and sometimes audibly, immersed in a virtual environment or augmented reality generated by a TBR platform. A headset may include a multitude of sensors for detecting a user's actual environment and the user's real-time interaction in that actual environment (such as the position and movement of the user's hand, and/or the position and movement of the user's head, and/or the position and movement of a controller held by or worn by the user, etc.). The tracking sensors, which may be integrated into the headset or may be external to the headset, depending on the particular configuration of the XR system, are leveraged generally to monitor a user's head movement and limb/body positions in real-time, sometimes even including eye movements, gaze direction, and foci-related data, allowing the virtual environment to update in response to, and in view of, the data accordingly.

An HMD also, inevitably, includes a screen or display component for rendering visual content for consumption by the user/wearer of the headset, as would be understood by one of ordinary skill in the art of XR systems. In some headset embodiments, the display component may be a liquid crystal display (or the like) that is an integrated part of the headset, while in other embodiments the headset may be configured to couple to a smartphone or other handheld device such that the smartphone provides the display component to the user by and through the headset to which it is coupled. Notably, in HMD embodiments that leverage a smartphone, sensors in the smartphone, as opposed to in the HMD or separately worn by the user, may be used to measure and track user movements.

The headset may also include one or more processors or chips configured and operable to process an immersive multimedia workload, as would be understood by one of ordinary skill in the art. As will become clear from the disclosure herein, the immersive multimedia workload may include one or more TBR therapies according to a TBR platform. The processors may be integral to the headset or, in some embodiments, may be remote from the headset and communicatively coupled to the components residing in the headset via wireless communications links or the like. The processor(s) may be in the form of central processing units, graphical processing units, application-specific integrated circuits, systems on a chip, or the like (as also defined above). Processors in an XR system may be responsible for any number of various functions including, but not limited to, processing data from sensors, rendering graphics, and managing the overall system.

Moreover, a headset of an XR system may include a camera subsystem or computer vision (“CV”) subsystem configured and operable to capture a user's real-world environment so that virtual objects may be overlaid on the image of the real-world environment, thereby creating an augmented environment with which the user may interact. For instance, and as would be understood by one of ordinary skill in the art of XR systems, a camera subsystem may be used to capture and render via the headset's display component a video stream of a user's living room such that virtual objects generated by the system may appear to reside in the actual room. As another example, a CV subsystem may be used for visual-inertial mapping, place recognition, and geometry reconstruction to establish the location of a user's hand in relation to other objects (real or virtual) within a given space. In this way, a camera and/or CV subsystem or module may be an important input device to a given XR system configured for use with a TBR platform according to the solution. Other input devices or controllers typical in an XR system may be handheld devices comprising directional tracking sensors (e.g., gyro sensors), or smart gloves comprised of position sensors (e.g., sensors associated with a wearer's knuckles, or fingertips, or palm, etc.), scanners (radar, LiDAR, time of flight, infrared, etc.), temperature sensors, actuatable “buttons” for user input/function selection, etc.

As would be appreciated by those of ordinary skill in the art of XR systems, as well as users of such systems, the immersive multimedia experience delivered by an XR system through high-quality visuals, audio, and accurate movement tracking can deliver a feeling to a user of being fully present within the virtual or augmented environment presented by the system. The immersive experience, depending on the software platform driving the system, may include the ability to manipulate objects and interact with the virtual or augmented environment through user input devices (whether the inputs are passive or active in nature). And, some systems provide user feedback beyond content visually rendered via the display, such as tactile feedback, to further enhance the realism of the user's perception of the virtual or augmented environment. Notably, depending on the goal of the software platform, feedback devices may be used to generate either a positive or negative feedback in order to coerce or influence user behavior in response to the virtual stimuli. Examples of desired user behavior could be linked to or derived from any number of patient specific therapies such as practicing activities of daily living, work conditioning, sport specific training, etc. Embodiments of a TBR platform may leverage any one or more of these feedback devices that may be present in an XR system.

It is envisioned that feedback mechanisms may be built into the solution itself. One such example pertains to eye tracking. To state the obvious, using a physical controller in XR may be quite difficult, if not altogether impossible, for amputee users but also for many nonamputee users such as those with nerve injury(s), stroke, CRPS, etc. To combat this reality, eye tracking may be employed by certain embodiments of the solution in order provide means for amputee users to manipulate objects in XR. Advantageously, certain embodiments of the solution improve the effectiveness of eye tracking for amputee users by employing eye tracking in combination with prompts and timers to perform and confirm certain actions. For example, a cursor or other identification item may be displayed on the screen at the position corresponding to the center of the user's gaze. If the user directs their gaze over an interactable object or button, then such action may inadvertently trigger an event or progression. To protect against inadvertent actuation of a function within the XR environment by a user employing a gaze-based controller, embodiments of the solution employ a second step in the form of a required confirmatory action or actions after the initial object or button is interacted with by gaze. For example, directing the gaze over an object or button could start a timer and the user would need to hold their gaze on the object or button for the duration of the timer in order to confirm their intent to actuate/select the object or button; or, once an object or button was activated by an initial gaze then a secondary object, button or prompt may arise that would also need to be interacted with to complete the desired action. Depending on embodiment, other feedback mechanisms may come in the form of combination actions, with one action being performed in the virtual environment and another confirmatory action or actions, being performed outside of the virtual environment. An example would be to direct gaze over an object or button presented in the XR environment and then to perform a confirmatory action outside of the XR environment to complete the progression. The confirmatory action outside of the XR environment may, for example, come in the form of audio (i.e. saying “yes/no”), direct input (press “A” for yes or “B” for no), or indirect input such as performing a certain movement or gesture (including activating or moving a phantom limb or prosthetic in specific way or ways through an external prosthetic, myoelectric sensor or neural monitoring input, etc.), etc. Such in-solution feedback mechanisms may ensure that physical disability or deformity would not prevent a user from interacting with the system.

Along these lines, it is an advantage of certain embodiments of the solution that an input sensor(s) of a myoelectric band operable to detect myoelectric impulses in an amputee's limb, residual limb/stump, or chest/trunk (or other sources such as EEG activity, or brain/spinal cord activity, or the like) may be leveraged to record and map an amputee's myoelectric responses to stimuli presented to the user in a virtual or augmented reality. In this way, data collected by embodiments of the solution may be used to inform a targeted muscle reinnervation (TMR) surgery that smartly maps a nerve reassignment to a complementary muscle(s) or muscle group that would be easily monitored with myoelectric sensors in a myoelectric prosthesis, advantageously preempting any need for the surgeon to “guess” at which nerves to “reassign” to corresponding muscle(s) or muscle groups to provide the desired functionality of the chosen prosthesis and, further, improving the likelihood of successful and efficient use of the prosthesis post-surgery. Moreover, by detecting and monitoring which residual nerves an amputee user's brain wants to associate with a given functionality of a prosthetic limb (such as during guided motor execution in the virtual environment), embodiments of the solution may efficiently train an amputee to use a myoelectric prosthesis even before ever having been fit for it, especially if implemented before any significant cortical remapping (i.e. shortly after the injury or disease onset).

As would be understood by a surgeon with experience in TMR procedures, a portion of an intact “targeted” muscle may be denervated by cutting the nerve that usually controls the muscle. The muscle may then be reinnervated by connecting it to one of many potential neural targets that were involved in the amputation. In this way, neural stimuli from the reinnervated muscle may be used to control the specific functions and/or motions of a prosthetic. Reassigning the severed nerve to a new function not only helps prevent neuroma growth and normalizes the nerve signals that are sent to the brain (thereby reducing the pain signals that cause phantom limb pain), but also improves control and use of a myoelectric prosthesis, especially if the reassigned nerve had been previously identified and confirmed to naturally associate with a particular function of the prosthesis.

Certain myoelectric prosthetic devices are controlled through two electrical signals generated by the contraction of two muscles or group(s) of muscles in the amputee's residual limb (such as for a “close hand” and “open hand” binary control prosthetic), but other, more advanced, myoelectric prosthetics may include a plurality of sensors, each capable of being combined via pattern recognition or individually mapped to any given functionality of the prosthesis. As would be understood by a surgeon with experience in this area, embodiments of the solution disclosed herein may help identify remaining functional neuromuscular targets for TMR. This would help the surgical and treatment team to identity, pre-surgery, which of the patient's remaining neuromuscular structures are best suited for reinnervation in association with various functions of the desired myoelectric prosthetic. Along these lines, the various myoelectric sensors in the prosthetic that will be physically associated with the reinnervated neuromuscular structures may be preprogrammed to map to the correct prosthetic actuator/functionality.

Similar to the above, in other embodiments of the solution for a TBR platform, data analytics collected by a myoelectric band, other myoelectric data capture device or neural feedback device such as an EEG, may be used to inform a training exercise regimen that conforms a user's residual nerves to a prosthetic, instead of the other way around. In such embodiments, a virtual representation of a myoelectric prosthetic may be manipulated in response to myoelectric signals detected by the band, or other monitoring device, worn by the user. These devices could be in the form of surface or implantable monitors/electrodes, etc. In this way, a given myoelectric signal that would cause a real-life version of the prosthetic, if it were fitted to the user, to move according to a given one of its designed degrees of freedom may be taken as an input to cause the virtual representation of the prosthetic to “move” accordingly. With this type of visual feedback to the user, embodiments of a TBR system may be used to train the user on exactly which movements or activations of residual muscles map to given functional capabilities of a myoelectric prosthesis to which the user will later be fitted. This is especially important in the time period before the prosthetic is delivered to the patient, which can take months. Cortical remodeling begins immediately after injury/amputation, and as it progresses it becomes more difficult for patients to learn to use a prosthetic effectively. The TBR system allows the patient to begin training on the desired prosthetic immediately following amputation to both prevent cortical reorganization AND train them on how to properly use the desired prosthetic. Moreover, the creation of an XR training environment for different prosthetics allows the TBR system to evaluate the user's training data and make recommendations on which type of real-world prosthetic the user is ready to transition into, and thus, which prosthetic an insurance company should pay for. Regarding this, it is envisioned that the TBR system may also allow for the creation of static and dynamic training plans to progress users through training on the different types/models of prosthetics in different situations best suited for that specific type/model of prosthetic. It is understood that most amputees utilize multiple different types/models of prosthetics for different situations (work, sports, activities of daily living, etc.). It is further envisioned that the TBR training programs could be guided by a caregiver or an intra-application virtual therapist and informed by neural network machine learning, and or artificial intelligence, to train each user on the different types/models of prosthetics so that they understand when to use which prosthetic and transition seamlessly between prosthetics.

Returning to the description of an exemplary XR system that may be employed by a TBR platform, sensors on XR devices, such as computer vision sensors and/or movement sensors integrated into a headset, may work together to collect various kinds of data such as, but not limited to, audio from the user's real-world environment, acceleration of a device (such as a headset or a handheld controller), orientation of an object (such as a user's hand), and topographical data of the user's real-world environment. The data may be leveraged by the XR platform to map and find objects in the user's real-world, physical environment. Mapping the user's real-world space entails constructing three-dimensional, topographical representations of the user's environment to accurately recreate the environment virtually and situate the user within the virtual space. Understanding the user's environment involves identifying physical objects or surfaces in the user's space to help place virtual content generated by the platform and/or virtual representations of the user himself (such as a virtual representation of the user's hand) and/or other users interacting with the primary user via the platform (such as with avatars). In other embodiments, the virtual environment presented to the user may be completely virtual and unrelated to the user's actual, real-world environment.

To map and identify objects in the user's real-world environment, XR devices collect data across various sensors, such as light sensors, microphones, cameras, stereoscopic 3D cameras, depth sensors, computer vision sensors, LiDAR, and inertial measurement units (IMUs) for measuring movement and orientation of the user. When a given sensor is experiencing a performance problem or certain sensor data is not available (such as data associated with a hidden portion of a user's hand when presented in a given posture), an XR device may utilize other sensors to fill in the data gap or leverage algorithms to generate proxy data. For example, if photons from a depth sensor fail to indicate a user's hand posture, an XR platform may use an algorithm to fill in the sensory gap using pixels closest to the area where the depth sensor directed the photons.

Once an XR device such as a user headset has gathered data through its sensors, the device and XR applications may need to further process this data to map and identify objects in the user's physical space. For example, sensor data collected by an XR device may be subjected to an application that fuses the sensor data, using algorithms that combine data from various sensors to improve the accuracy of simultaneous localization and mapping (“SLAM”) and concurrent odometry and mapping (“COM”) algorithms. As would be understood by one of ordinary skill in the art of XR platforms, SLAM and COM algorithms map a user's surrounding area, including the placement of landmarks or map points, and help determine where the user should be situated virtually in the virtual environment. Some types of XR platforms leverage computer vision AI systems to identify and place specific objects within a virtual environment. These applications may also use machine learning models to help determine where to place “dynamic” virtual content-virtual objects that respond to changes in the environment caused by adjustments to the user's perspective. These mapping and object identification functions may also allow for shared experiences by multiple users. For example, in a TBR platform configured to administer a therapeutic session between an amputee user and a remote clinician, the user and remote clinician, via their avatars, could toss a virtual ball back and forth such that the amputee user employs a virtual limb to catch and/or throw the ball.

It is envisioned that embodiments of a TBR platform may enable XR devices to send mapping and environmental sensor data to other users, such as between an amputee user and a remote clinician user. For example, raw sensor data may be transmitted to the TBR platform to improve existing device functions, such as the placement and responsiveness of virtual content that the amputee user and clinician user interact with. An external server employed by a TBR platform may also process and relay users' location information collected by the user-associated XR subsystems, such as approximate or precise geolocation data, to enable shared experiences. For instance, an amputee user and a caregiver user could interact with each other's avatars to administer a TBR therapy to a phantom limb of the amputee user, with each using their own XR subsystems that recognize the placement and posture of the other in a virtual space. Moreover, a TBR platform administrator may be able to observe processed sensor and other analytical data generated by the users' interaction with the TBR platform.

Some XR technologies gather and process sensor data to enable controller-based and gesture-based interactions with physical and virtual content, including other users. Gesture-based controls allow users to interact with and manipulate virtual objects in ways that are more reflective of real-world interactions. Most devices use inertial measurement units and outward-facing cameras combined with infrared or LED light systems to gather data about the controller's position, such as the controller's linear acceleration and rotational velocity, as well as optical data about the user's environment (computer vision sensors). It is envisioned, however, that preferred embodiments of a TBR platform will leverage gesture-based controls, by employing XR devices that gather data about a user's remaining limb through outward-facing cameras and/or computer vision sensors on the user's headset.

As would be understood by those of ordinary skill in the art of XR, XR platforms use algorithms and machine learning models to provide controller-based and gesture-based controls. In controller-based systems, algorithms use data about the controller's position to detect and measure how far away the controllers are from the user's headset. This allows the user's “hands” to interact with virtual content provided in the virtual environment. It is envisioned, however, that TBR platforms may preferably leverage XR systems with gesture-based controls that utilize machine learning models, specifically deep learning models, to generate three-dimensional (“3D”) copies of a user's remaining limb, such as a hand or foot, by processing images of the limb in its physical-world and determining the location of its knuckles/joints or other useful physiological landmarks. That is, and as would be understood by one of ordinary skill in the art of XR technologies, deep neural networks may be used to predict the location of a user's hand as well as landmarks of the hand, such as its joints. These landmarks may then be used to reconstruct a pose or posture of the user's actual hand and fingers. The result is a 3D model in XR of the user's actual hand that includes the configuration and surface geometry of the hand. Use of external scanners and cameras allow the capture of size, texture and surface anatomy of the remaining/opposite limb(s) to guide the creation of a realistic 3D model of the missing limb in XR. This is important in certain embodiments of the solution as increased realism of the virtual representation of the missing limb allows for improved treatment effects especially when targeting cortical reorganization. Other methods for generating 3D copies of a user's limb, such as use of inverse kinematics, are understood by those of skill in the art. Regardless of how the position/gesture of a user's limb is calculated, a TBR platform, advantageously, may recognize gestures or postures of the user's remaining (“good”) limb and mirror the gesture by a virtual limb (i.e. a generated virtual hand) associated with the user's lost limb. In this manner, the TBR system may reconstruct and, thus, reanimate lost limbs in the XR environment in 3 dimensions and map movements and gestures to these reanimated limbs based on inputs from remaining limbs or other internal/external controls. Along the same lines, in users who have undergone multiple limb amputations, all limbs could be reanimated without a remaining “good” copy by using geometric inputs of the remaining stump(s) and specific patient factors to project an anatomically correct limb(s). These reanimated limbs may then be controlled in XR in 3 dimensions based upon the motion of the remaining limbs and/or stump(s) and inputs from myoelectric or other neural sensors. Advantageously, in such embodiments of the solution a multi-limb amputee otherwise unable to utilize mirror therapy but could use remaining musculature, neurologic, and/or myoelectric inputs to power anatomically correct limbs in XR to treat/prevent cortical reorganization, train on different prosthetics, and treat phantom limb pain. So, it is an advantage of certain embodiments of the TBR system that it enables mirror therapy type treatment(s) in patients who have undergone multiple limb amputations; something that is currently unavailable to amputees due to the inherent limitations of standard mirror therapy.

It is envisioned that embodiments of an XR system employed by a TBR platform may track any number and combination of a user's body movements. Body tracking captures eye movements or gazes (using inward facing cameras), facial expressions, and other body movements, which can be used to create avatars that reflect a user's reactions to content and expressions in real-time. As would be understood by one of ordinary skill in the art, avatars are a user's representative in a virtual or other artificial environment generated by an XR platform. XR platforms configured to track a user's body movements and map them to an avatar's movement, enable a TBR platform to provide more realistic interactions between an amputee user and a clinician user and/or caregiver user. For example, in a virtual environment whereby a remote clinician is coaching an amputee user through a TBR neuro-rehabilitation technique (such as a laterality recognition exercise), a realistic avatar of the amputee user may display facial and other body movements that can be perceived by the clinician and used to modify the therapeutic exercise. As outlined above, to depict a user's reactions and expressions on their avatar, XR platforms need data about the eyes, face, and other parts of the user's body. A device may use IMUs and internal-and outward-facing cameras and other sensors to capture information about the user's head and body position, gaze, and facial movements. XR devices may also use microphones to capture audio corresponding with certain facial movements and/or mouth shapes (“visemes”), as a proxy for visuals of the user's mouth when the latter is unavailable. For instance, the sound of abrupt inhaling may cause an avatar to show behavior indicative of sudden pain.

In addition to avatar creation and operation, XR technologies leveraged by TBR platforms may monitor gaze and pupil dilation, motion data, and other information derived from the user's body to generate behavioral insights. Certain XR platforms employed by a TBR platform according to the solution may be capable of using sensor data that is generated in response to stimuli and interactions with content to make inferences about a user's physical, mental, and emotional conditions in response to TBR therapies, such as guided motor imagery (i.e., imagined movements/explicit motor imagery), experienced through the virtual environment. When combined with information processed by other sensors, such as brain-computer interfaces, these body-derived data points may contribute to the creation of unique and granular individual profiles and insights into the user's health as they engage with the TBR platform.

In view of the above, it will be understood that XR systems may be driven by embodiments of the present solution for a TBR platform to deliver unique therapies to a user who is an amputee and experiencing phantom limb pain (or at-risk to experience phantom limb pain). Turning now to the figures, an embodiment of the solution for a TBR platform is described in more detail.

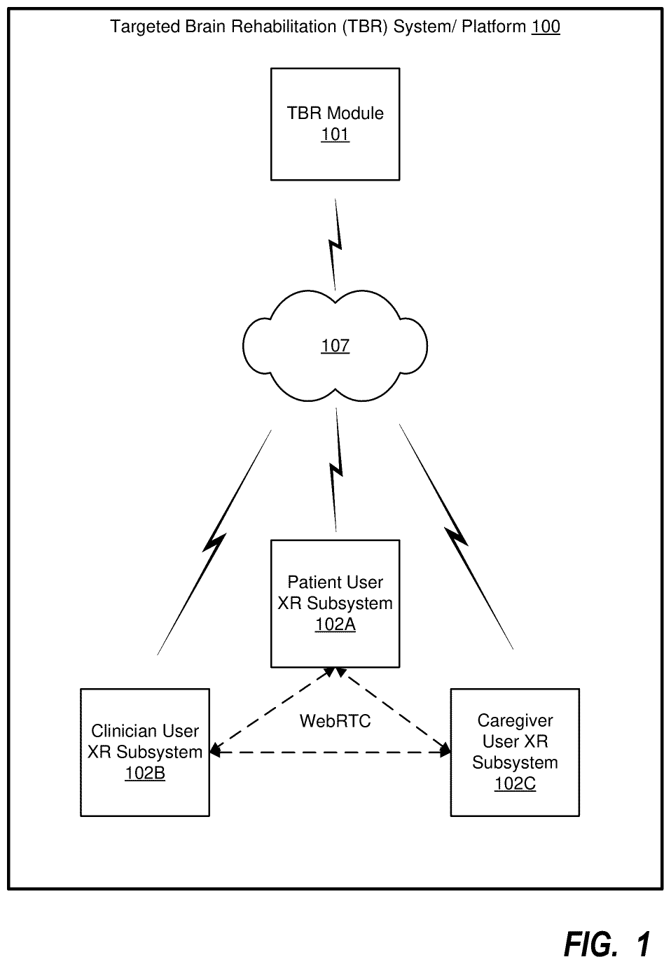

FIG. 1 depicts a high-level, functional block diagram of a targeted brain rehabilitation platform (“TBR platform”) according to the solution. The TBR platform 100 is in communication via a wired or wireless link with an external network 107, which can be a public or private network or a combination of public and/or private networks. In a preferred embodiment, XR user subsystems 102 may include browsers enabled with javascript APIs configured for real-time, peer-to-peer communication of audio and/or video content. It is envisioned, however, that alternative embodiments of the solution may leverage web sockets for communication between the user devices via and through a peer-to-server arrangement with the TBR module 101, as would be understood by one of ordinary skill in the art of online communication protocols and methodologies.

As will become clear from subsequent figures and description, the TBR module 101, in cooperation with one or more user XR subsystems 102, may generate a virtual environment configured for hosting TBR sessions in which TBR therapies are administered. The TBR module 101 may be in the form of a server or “desktop” computer hosted by an administrator. In some embodiments, the TBR module 101 may be in the form of an XR subsystem itself or gaming platform or any other computing device configured and operable to host a TBR session. Moreover, in some embodiments, the TBR module 101 may be distributed across multiple computing devices.

In the FIG. 1 illustration, three user XR subsystems 102 are depicted, although it is envisioned that any number or combination of subsystems 102 and/or TBR module 101 may be employed within a given TBR platform 100. It is an advantage of certain embodiments of the solution that users may be remote from the TBR module 101 and/or remote from each other while engaged in a TBR session. A patient user XR subsystem 102A, however, is central to most embodiments of the solution, as the subsystem 102A is the particular subsystem used to deliver a multimedia immersive experience to a patient during a TBR session. Clinician user XR subsystem(s) 102B and caregiver user XR subsystem(s) 102C may be associated with clinicians and caregivers, respectively, who may participate in a TBR session. In this way, a clinician or other caregiver (such as a patient user's family member) may virtually interact with the patient user within a virtual environment to administer, or participate in administering, a TBR therapy.

The user XR subsystems 102 may be in communication via a WebRTC protocol, as previously described, although not all embodiments of the solution for a TBR platform require WebRTC architecture. It is envisioned, for example, that embodiments of a TBR platform may be comprised completely within an “at home” XR system such that the user XR subsystem 102A is in communication with a host computer (such as a gaming console) in the role of the TBR module 101.

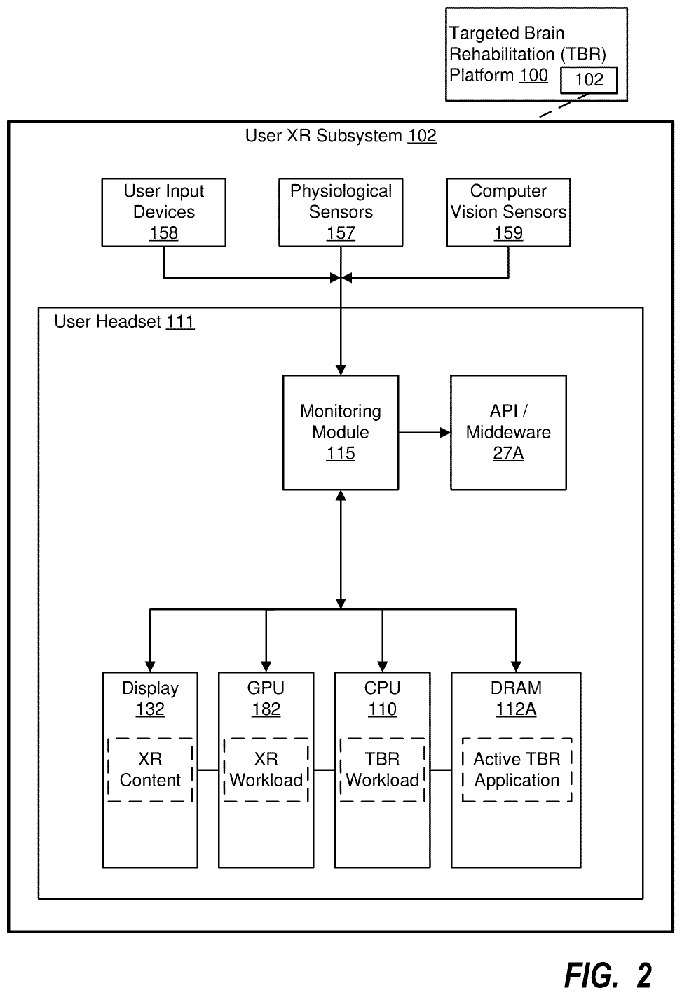

FIG. 2 depicts a functional block diagram of an exemplary user's XR subsystem 102 that may be employed within a TBR platform 100. As previously described, XR subsystem(s) 102 may be associated with a clinician user and/or a caregiver user, as well as a patient user, participating in a TBR session administered by the platform 100.

An XR subsystem 102 may be configured and programmed to render an immersive multimedia output to its user wearer. The XR subsystem 102 may comprise user input devices 158, physiological sensors 157, and/or computer vision sensors 159 in communication with (and sometimes integrated into) a user headset 111. The headset 111 may have a set of left and right optical lenses through which the user may visually experience a multimedia output of XR content rendered on the display 132. In some embodiments, the headset 111 may be configured to interface with a portable computing device, such as a smartphone, such that the smartphone provides the display 132 and other components. In such an embodiment of a headset 111, the display 132 may be juxtaposed and mechanically fixed to the front of the headset 111 such that the multimedia output(s) are aligned with the left and right optical lenses.

With the headset 111 mounted to the user of the XR subsystem 102, motion of the user's head may be recognized by motion sensors 157 and the multimedia output of the headset 111 reconciled therewith. The physiological sensors 157 may further include, depending on embodiment, any number of sensors including, but not limited to, eye-tracking sensors, myoelectric sensors, EEG sensors to interpret brain wave patterns (for mapping both imagined and executed movements to corresponding virtual actions), pressure-sensitive gloves, etc. The user input devices 158 may include, but are not limited to including, adaptive controllers, universal adapters for interfacing with a user's prosthetic, etc. And, the computer vision sensors 159 may be infrared sensors, LiDAR sensors, camera subsystems, or other sensing technology useful for capturing a user's immediate environment, including a hand or other limb of the user, and generating signals representative thereof and useful for the TBR platform 100 to create a virtual environment for a TBR session.

The headset 111, may essentially include an on-chip system for executing and rendering an immersive multimedia workload associated with a TBR session or therapy. An active TBR application associated with the subsystem 102 (shown stored in the DRAM 112A) may be in execution by various processing components such as, but not necessarily limited to, the CPU 110, GPU 182 and LCD display 132. As would be understood by one of ordinary skill in the art, workloads associated with the active application may be processed by the processing components to generate an immersive multimedia output and user experience.

Additionally, a monitoring module 115 (which may include CPU 110 and/or GPU 182, among other components illustrated or not illustrated) may track all inputs and outputs of the subsystem 102 such that the collected data may be shared, as appropriate or required, with other user XR subsystems 102 and TBR module 101 cooperatively participating in a TBR session. An API/Middleware module 27A may be the means by which data tracked and collected by the monitoring module 115, as well as executable instructions and application workloads, are shared with other devices of the platform 100, as would be understood by one of ordinary skill in the art.

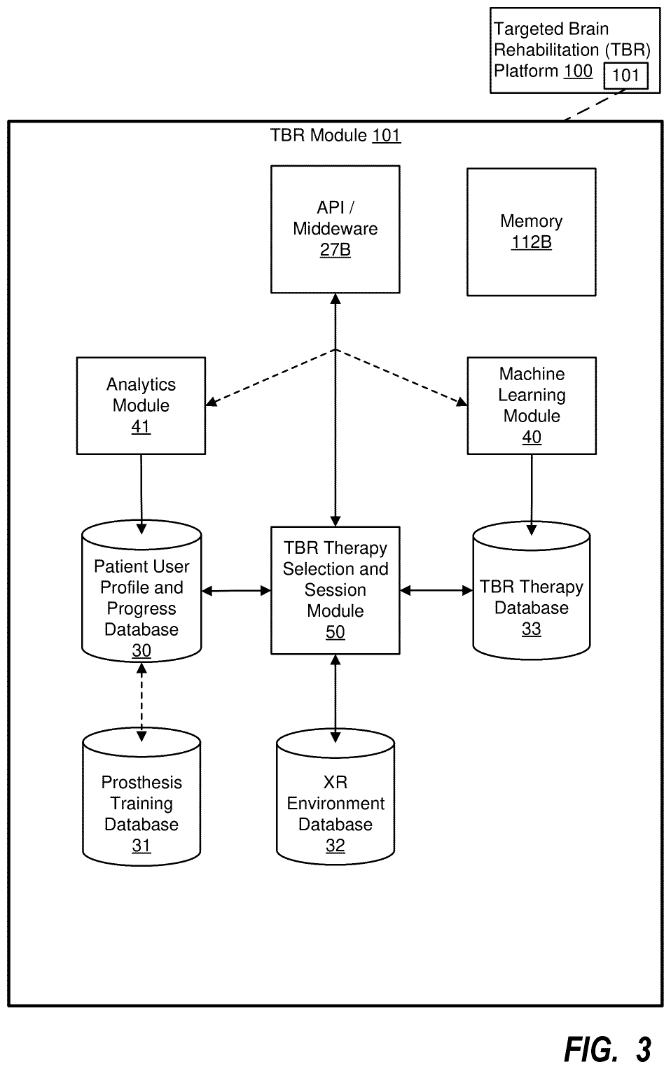

FIG. 3 depicts a functional block diagram of an exemplary TBR module 101 within a TBR platform 100. As previously described, the TBR module 101 is the “hub” of a TBR platform solution. API/Middleware 27B may enable the TBR module 101 to share data and instructions across the platform 100 with other devices, such as user XR subsystems 102. Memory 112B may store executable instructions associated with the TBR Therapy Selection and Session module 50 such as for generating an XR environment, hosting a TBR session, administering TBR therapies, etc. The memory 112B may also store executable machine learning algorithms for the ML module 40, analytics monitoring algorithms for the analytics module 41, and historical data for any one or more of the databases 30, 31, 32, 33.

The TBR module 101, in communication with the various user XR subsystems 102, may generate and render a virtual environment for hosting a TBR session. The TBR therapy selection and session module 50 may query and update the various databases 30, 31, 32, 33 as required in response to use of the TBR platform 100 by the various users. The XR environment database 32 may store applications and data useful for generating and rendering an XR environment. The patient user profile and progress database may store user profile information and therapy progress data. The user profile information may include any number of data such as, but not limited to, personal identification data, prosthetic type, health/medical data, user preferences, user XR subsystem specifications, etc. The therapy progress data may be associated with a particular user for tracking progress of previous TBR sessions including user feedback during the session with regards to pain level, ease of task completion, etc.

An analytics module 41 may work to monitor a patient user's involuntary inputs during TBR sessions, as generated by the physiological sensors 157 such as a myoelectric band. In this way, the analytics module 41 may work to populate the prosthesis training database 31, as well as portions of the progress database 30, to track a patient user's proficiency in using a virtual prosthetic representative of an actual prosthetic with which the user may be later fitted.

The TBR database 33 may house data and applications associated with the various TBR therapeutic methodologies available to a user of the TBR platform. The TBR selection and session module 50 may work with the TBR Therapy database to query and upload various TBR exercises or therapies as a patient user advances or navigates the functionalities of the TBR platform 100. A machine learning module 41 may work to update and refine the data and applications of the TBR therapy database 33 to customize them to a given patient user and/or improve them for the benefit of a patient user.

The various databases 30, 31, 32, 33 and modules 40, 41, 50 of the exemplary TBR module 101 illustrated in FIG. 3 may be combined into a single module or database, as the case may be, and depending on the embodiment. The databases and modules are depicted and described separately for ease of understanding, as would be acknowledged by one of ordinary skill in the art. The TBR module 101, in cooperation with the user XR subsystems 102, leverage their various sensors and input devices, processing components, output devices, stored data, and applications to administer TBR therapies in TBR sessions hosted by and through an XR environment.

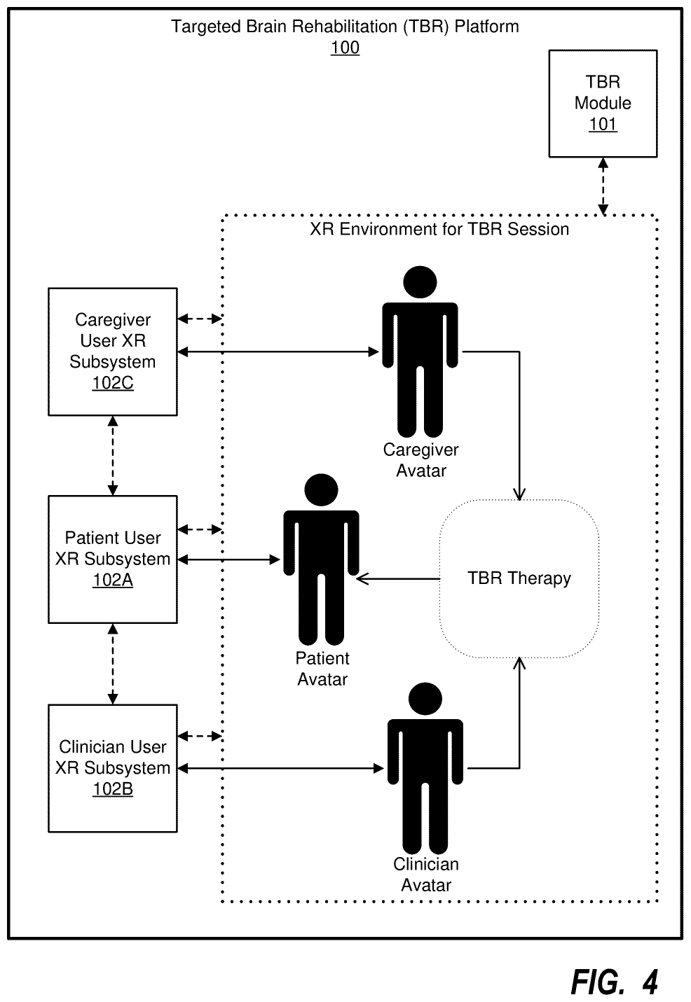

FIG. 4 illustrates an XR environment created by a TBR platform for hosting a TBR session and administering a TBR therapy. The TBR module 101, in cooperation with the user XR subsystems 102, generates and provides an XR environment within which the various users can interact by and through the user XR subsystems 102. Each user may have his/her avatar presented within the XR environment. A TBR therapy may be administered to the patient user associated with the subsystem 102A, as the avatar of the patient user engages with the avatars associated with the clinician user and/or the caregiver user. By and through the XR subsystems 102B, 102C, the clinician and/or caregiver users may leverage their avatars within the XR environment to engage with the patient user's avatar such that a TBR therapy is administered to the patient user. Notably, users associated with other XR subsystems 102B, 102C may engage with and help and guide the patient user of XR subsystem 102A within the XR environment to facilitate any of the functions offered by the TBR platform 100. Not all embodiments of a TBR platform 100 require active participation from clinician and/or caregiver users; however, it is an advantage of some embodiments that clinicians and/or caregivers geographically remote from a patient user may engage with the patient user through the platform.

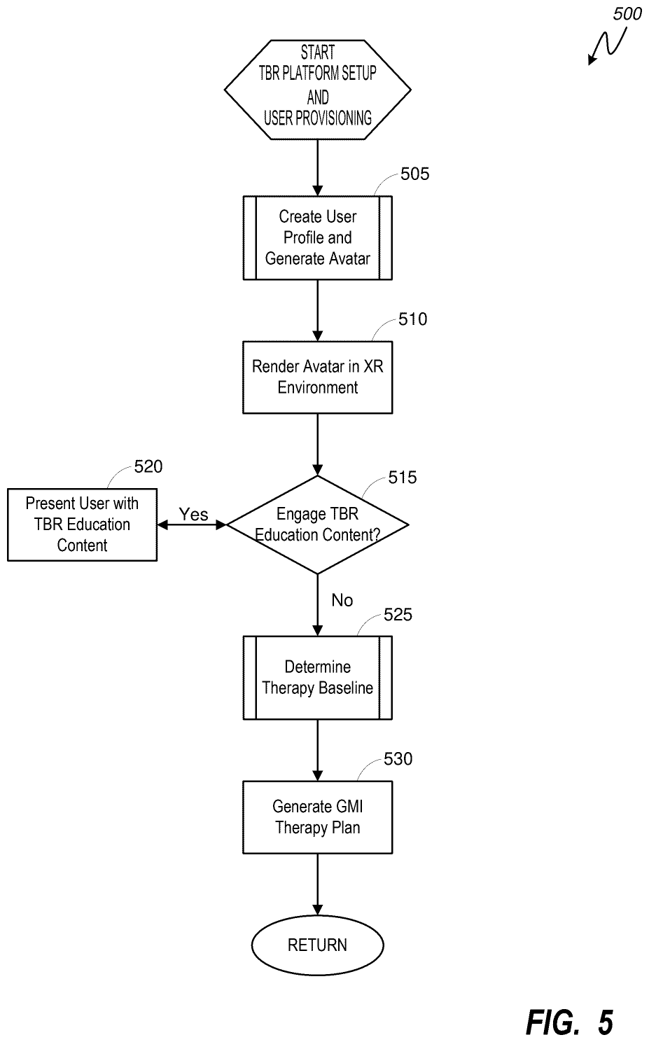

FIG. 5 is a flowchart illustrating an exemplary method 500 for provisioning an exemplary TBR platform 100 for use by a patient user associated with a XR subsystem 102A. The method 500 begins at block 505 where users (patient, clinician, caregiver, etc.), using a XR subsystem 102 and/or TBR module 101, create unique user profiles and select an avatar. The avatar may be customizable to accurately represent a given user in some embodiments, including virtual representation of prosthetics and the like. Once a user profile is established and an avatar for the user generated, the method may continue to block 510 where the avatar is rendered within the XR environment and becomes the user's virtual means for interacting with the TBR platform, as would be understood by one of ordinary skill in the art of XR technology.

The method 500 may include a decision block 515 whereby the user is given an opportunity to engage with educational content to train or educate the user on the functionality of the TBR platform 100, the XR subsystems 102, the TBR therapies, or the like. Depending on embodiment, a user may be presented with an opportunity to engage or skip the educational content at every login event. If the user elects to view educational content, the method may present the content at block 520. When the user has completed viewing of the educational content, the method may continue from decision block 515 to process block 525 to determine a baseline metric for the user. A baseline metric may be established at the user's first session on a TBR platform 100 such that data later tracked and collected as the user advances through TBR sessions, exercises, and therapies can be compared to the baseline to determine user progress and improvement. The baseline data may include, but is not limited to including. myoelectric data, pain levels, user survey inputs, accuracy of laterality recognition, efficiency at movement of a virtual prosthetic, etc.

With a therapy baseline determined at block 525, the method 500 may continue to block 530 where, in view of the collected baseline data, the TBR platform 100 may generate an initial Targeted Brain Rehabilitation (TBR) therapy plan. With a TBR therapy plan established, the various users may employ the platform 100 to administer the plan to the patient user by allowing the patient user to participate in TBR sessions wherein TBR therapies and exercises are deployed in an XR environment.

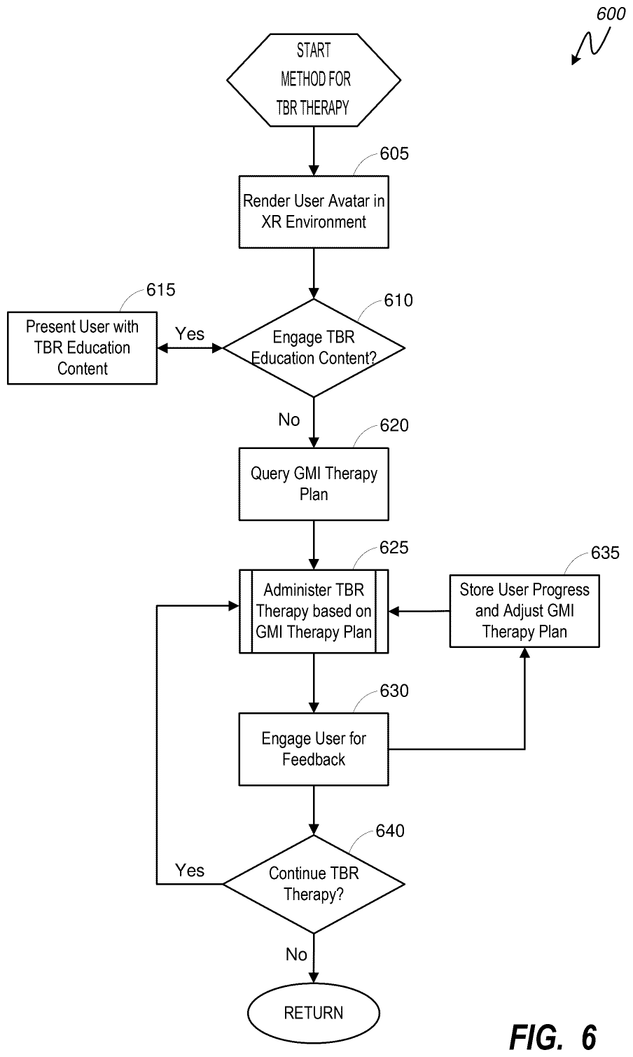

FIG. 6 is a flowchart illustrating an exemplary method 600 for providing TBR therapy sessions to a patient user of a TBR platform 100 according to the solution. Method 600 may be executed after the previously described exemplary method 500 has be executed for provisioning an exemplary TBR platform 100 for use by a patient user associated with a XR subsystem 102A. That is, method 600 may be the method deployed when a patient user who has previously established login credentials, avatar, therapy baseline, etc. returns to the TBR platform 100 for TBR therapy.

Beginning at block 605, the patient user's avatar may be rendered in an XR environment. The XR environment may be selectable by the patient user (or another user participating in a given TBR session). For example, it is envisioned that a user may select an XR environment reminiscent of a beach, or a living room, or any other environment conducive to a successful TBR therapy experience. In some embodiments, the XR environment may be an augmented reality environment that overlays digital content over a real-life environment as seen through XR glasses or the like.

Returning to the method 600, at decision block 610, the patient user may be given the opportunity to engage with educational content on TBR therapy, the TBR platform, and/or other relevant content. If the user elects to engage with the educational content, such as by actuating a virtual “button” presented in the XR environment or leverage an input device 158 (such as a handheld controller) or use a predefined gesture recognizable by computer vision sensors 159, or provide an audible command received by a physiological sensor 157, or take some other action recognizable by the XR system and TBR platform 100, the method 600 may proceed to block 615 and present the relevant content to the user in the XR environment.

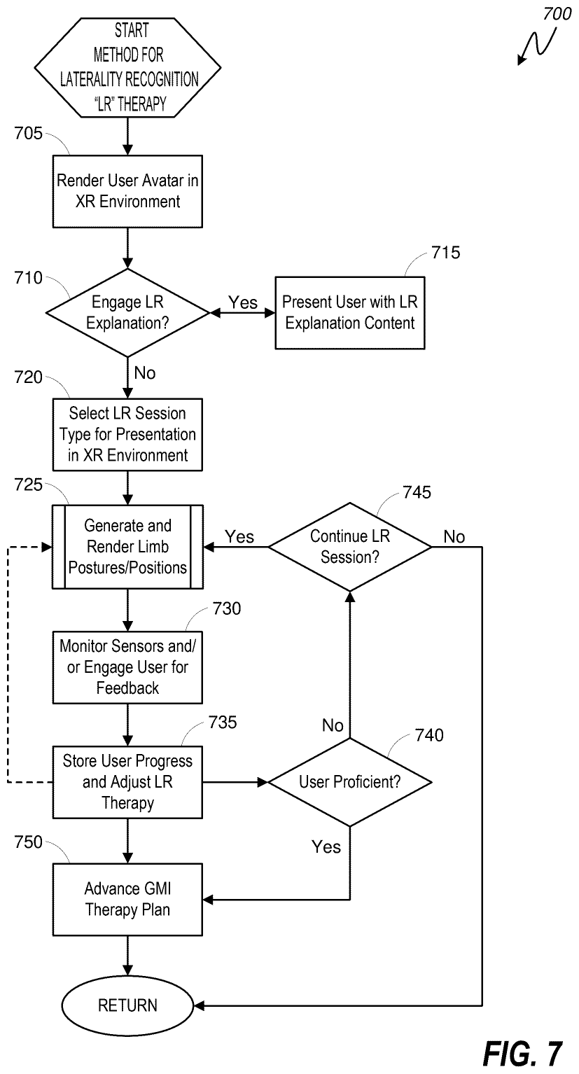

The method 600 continues from decision block 610 to block 620 where a previously generated TBR therapy plan may be queried from the TBR therapy database 33. At process block 625, the patient user may be engaged in the XR environment with a TBR therapy exercise determined by the TBR therapy plan. Exemplary TBR therapy exercises that may be deployed in the XR environment according to a TBR therapy plan will be described in connection with the flowcharts of FIGS. 7-9 that follow.

After engaging the patient user in the XR environment with a TBR therapy exercise, at block 630 the patient user may be surveyed for user feedback on the experience of the TBR therapy exercise. For example, the patient user may be surveyed for a pain rating that may be used to adjust the TBR therapy plan. In some embodiments, physiological sensors 157 may be leveraged while the patient user is engaged in the TBR therapy exercise to determine passive feedback from the patient user. In some embodiments. clinician users and/or caregiver users, leveraging their own avatars to interact with the avatar of the patient user in the XR environment may provide the TBR platform 100 with the feedback envisioned at block 630.

Based on the user feedback of block 630, the method 600 may advance to block 635 where the TBR therapy plan is adjusted and updated for future sessions in view of the feedback. The ML module 40 previously described may use the feedback inputs to smartly tune a TBR therapy plan or, in some embodiments, the clinician user may adjust the TBR therapy plan in view of the feedback. In these ways, the TBR platform 100 may continuously customize a TBR therapy plan such that the therapeutic experience for a given patient user is optimized.

With user feedback collected at block 630 and the various databases described in FIG. 3 updated, the method 600 may advance to decision block 640 where the patient user is given the option to continue with the TBR session and therapy plan or exit the TBR session. If the user elects to continue with the session, the TBR platform 100 may return to process block 625 where an updated TBR therapy plan is queried for a next TBR exercise. If the user elects to discontinue the session, the method 600 ends.

FIG. 7 is a flowchart illustrating an exemplary method 700 for providing TBR therapy in the form of a laterality recognition therapy to a patient user of a TBR platform 100 according to the solution. The method 700 is an example of a TBR therapy that may be a part of a TBR therapy plan and administered to a patient user of a TBR platform in an XR environment.

Turning to the method 700, the steps of block 705, decision block 710, and block 715 may be steps similar to those previously described relative to blocks 605, 610, and 615. The educational content available for presentation in the XR environment may be targeted to laterality recognition therapy. At block 720, a TBR session for laterality recognition exercises may be selected, generated, and rendered in the XR environment. The method 700 advances to process block 725 where the TBR platform 100 generates and renders limb postures/positions for the patient user to view. The limb postures may be simply presented using an “automated” avatar or the limb postures may be presented by an avatar associated with another user of the TBR platform who is participating in the session. The patient user may be asked to identify the laterality of the presented limb posture (e.g., is it a left hand or a right hand?) and make an input representative of the answer. Physiological sensors 157 and/or input devices 158 and/or computer vision sensors 159 of the patient user's XR subsystem 102A may be monitored and leveraged to collect user feedback at block 730.

Based on the feedback, at block 735 the method 700 may store data associated with the patient user, such as progress and success rate for given laterality recognition exercises conducted in the XR environment and update the TBR therapy plan accordingly relative to laterality recognition. In this way, the TBR platform 100 may continuously improve and adjust the TBR therapy plan such that the next execution of process block 725 works to present the patient user with an optimized laterality recognition exercise for the patient user.

After each exercise, the TBR platform 100 may determine at decision block 740 whether the patient user is sufficiently efficient at laterality recognition and, if so, at block 750 may advance the TBR therapy plan, giving the patient user access to further TBR functionality of the TBR platform. If the patient user has not achieved a suitable level of success with laterality recognition, the method 700 may advance from decision block 740 to decision block 745 where the method 700 may continue from process block 725 or end the TBR session.

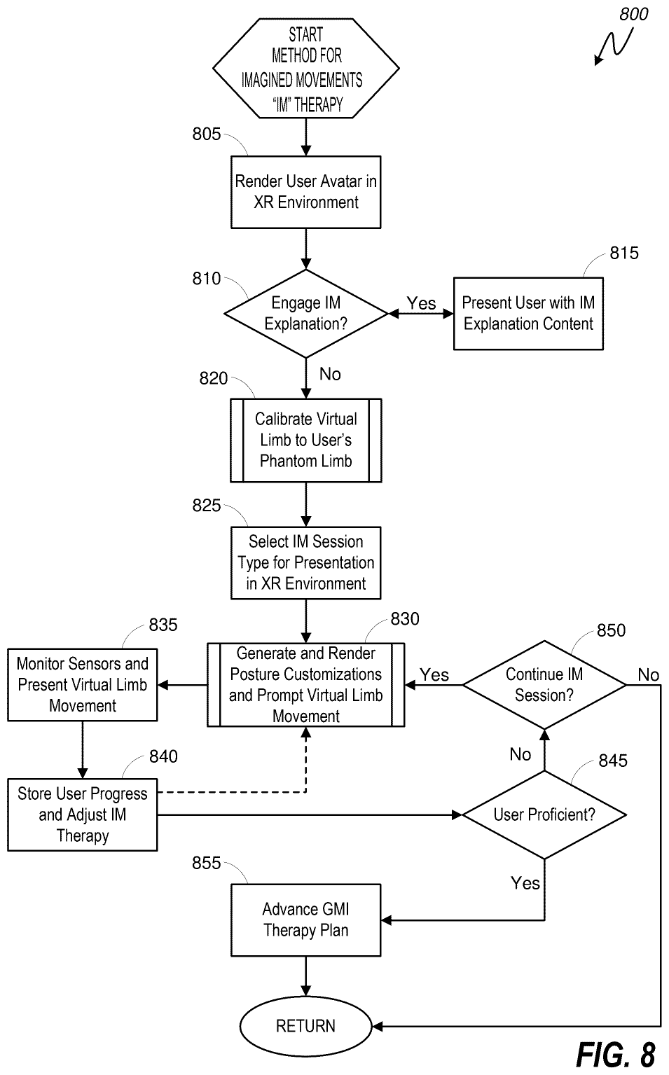

FIG. 8 is a flowchart illustrating an exemplary method 800 for providing TBR therapy in the form of an imagined movements (“IM”) therapy to a patient user of a TBR platform 100 according to the solution. The method 800 is an example of a TBR therapy that may be a part of a TBR therapy plan and administered to a patient user of a TBR platform 100 in an XR environment.

Turning to the method 800, the steps of block 805, decision block 810, and block 815 may be steps similar to those previously described relative to blocks 605, 610, and 615. The educational content available for presentation in the XR environment may be targeted to imagined movements (“IM”) therapy. At process block 820, a virtual representation of a patient user's missing/phantom limb may be rendered via the patient user's avatar and calibrated in view of myoelectrical signals captured by a myoelectric band (a physiological sensor 157). The myoelectrical signals may be generated by the patient user in response to stimuli presented to the user in the XR environment. For example, the patient user may be asked to imagine specific physiological movements so that the TBR platform may record myoelectrical signals that are a signature of the patient user's muscle and brain activity. In this way, a unique myoelectrical signal pattern of the user may be mapped to the presented stimuli (e.g., imagine your hand in a fist) such that the virtual limb can be calibrated to the myoelectrical patterns and, by extension, the TBR platform 100 can accurately interpret the patient user's imagined movements. Notably, as the patient user subsequently engages in IM therapy exercises, the ML module 40 may continually monitor and improve the accuracy of the calibration. It is an advantage of embodiments of the solution that the user's virtual environment may be completely “blacked out” such that only the stimuli intended for the user is presented to the user (whether that stimuli is visual or audial or both), thereby mitigating or altogether preventing unintended stimuli that may distract the user and undermine the therapeutic efficacy (such as is often experienced in traditional, in-person therapeutic environments).

With the virtual limb calibrated to the patient user's myoelectrical signal patterns, the method 800 may proceed to block 825 where an IM therapy session is configured. The IM therapy session may deploy any number of IM therapy exercises to train the patient user to move the virtual limb with only brain activity and any remaining musculature monitored by the myoelectric sensor band and/or EEG sensors. Continuing to process block 830, the method 800 may ask the patient user to move the virtual limb to match a given position, such as a limb position presented using the avatar of a clinician user, for example. Based on sensor data collected by the TBR platform 100, the method 800 may present the virtual limb in a posture or movement pattern associated with the patient user's myoelectric and/or EEG signal patterns at block 835, thereby giving the patient user visual feedback that he can use to “retrain” his brain and residual limb. At block 840, the patient user's progress and relative success rate can be stored and used to adjust the IM therapeutic approach.

If/when the patient user becomes proficient with movement of the virtual limb (or has met some other predefined threshold for advancement), at decision block 845 the method 800 may update/advance the overall TBR therapy plan for that patient user at block 855. If the patient user is less than proficient at decision block 845, the method 800 may proceed to decision block 850 where the patient user may elect to continue with IM therapy at process block 830 or end the TBR therapy session.

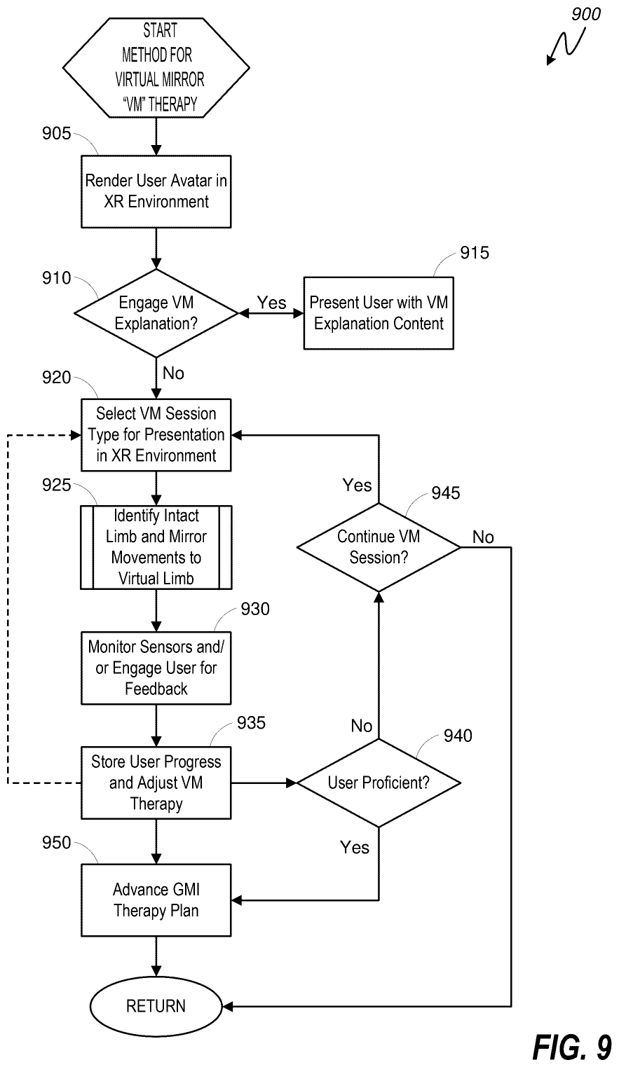

FIG. 9 is a flowchart illustrating an exemplary method 900 for providing TBR therapy in the form of a virtual mirror (“VM”) therapy to a patient user of a TBR platform 100 according to the solution. The method 900 is an example of a TBR therapy that may be a part of a TBR therapy plan and administered to a patient user of a TBR platform 100 in an XR environment.

Turning to the method 900, the steps of block 905, decision block 910, and block 915 may be steps similar to those previously described relative to blocks 605, 610, and 615. The educational content available for presentation in the XR environment may be targeted to virtual mirror (“VM”) therapy. At block 920, a virtual mirror therapy session may be instituted to deliver VM therapy exercises to a patient user engaged with the XR environment. At process block 925, the patient user's intact limb may be recognized via computer vision sensors 159 or other means of an XR subsystem 102A, as would be understood by one of ordinary skill in the art of XR technology. Using a virtual representation of the patient user's intact limb, the TBR platform 100 may cause a virtual representation of the patient user's missing/phantom limb to mirror or mimic the movements of the intact limb, thereby promoting neuroplasticity in the patient user's brain and combatting phantom limb pain.

Sensors monitored at block 930 may be indicative of the patient user's feedback resulting from engaging with the VM therapy exercise or, in some embodiments, the patient user may be queried for feedback to the TBR platform 100. At block 935, the TBR platform 100 may store the patient user's progress in the VM therapy phase and use the progress data to improve and update the VM phase of the TBR therapy plan. If the patient user is proficient at VM therapy at decision block 940 (or has met some other predefined threshold for advancement), the TBR platform may advance the TBR therapy plan at block 950. Otherwise, the method 900 may advance the patient user to decision block 945 where the VM therapy session may continue back at process block 920 or end the session.

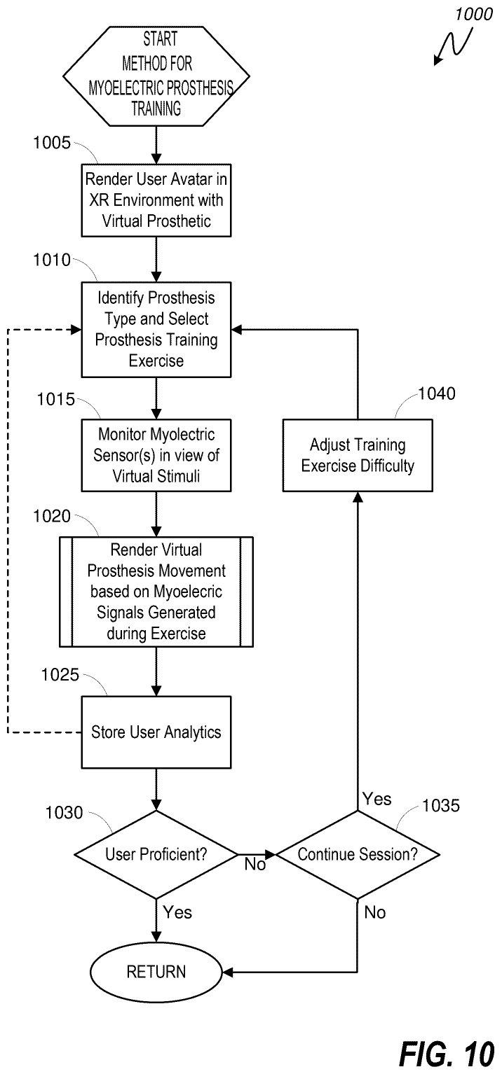

FIG. 10 is a flowchart illustrating an exemplary method 1000 for myoelectric prosthesis training of a patient user of a TBR platform 100 according to the solution. It is envisioned that certain steps of the method 1000 may be executed by a TBR platform simultaneously with execution of other TBR therapy exercises and methodologies, such as mirror therapy or imagined movements therapy. At block 1005, a patient user's avatar may be rendered in an XR environment. The avatar may be rendered with a virtual prosthesis that is representative of a real-life prosthetic having given specifications and defined degrees of freedom for movements/motions. As such, at block 1010 the particular real-life prosthesis, or at least certain specs of the real-life prosthesis, may be identified to the TBR platform 100 and used to determine an appropriate myoelectric (or other class) prosthesis training exercise.

At block 1015, myoelectric signals generated by the brain and musculature of the patient user in response to stimuli presented in the XR environment may be monitored by EMG and/or EEG sensors and used by the TBR platform 100 at process block 1020 to manipulate the virtual representation of the myoelectric prosthesis. At block 1025, as the patient user continues to engage with the training exercises, progress and success data is collected and used to advance the training. When at decision block 1030 it is determined that the patient user is proficient at use of the virtual prosthetic or has met some other predefined threshold for advancement (thus, indicating that the user is ready to be fitted with the real-life prosthetic), the method 1000 may conclude. If the user is not fully proficient, however, the method 1000 may advance to decision block 1035 where the user may elect to continue with the myoelectric prosthesis training or end the TBR session.

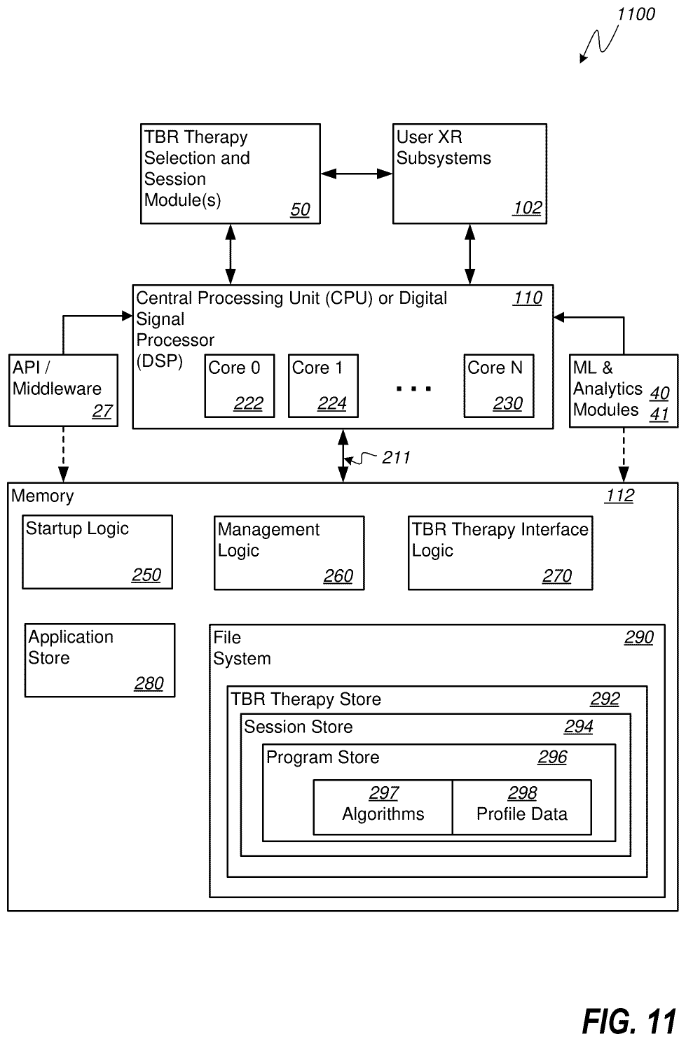

FIG. 11 is a schematic diagram 1100 illustrating an exemplary software architecture of the TBR platform of FIG. 1 for providing TBR therapy sessions via an immersive multimedia workload. Any number of algorithms may form or be part of a TBR therapy within a TBR therapy plan that may be executed by the various modules of the platform 100, as previously described. The modules 40, 41, 50, 115, 101, including the processing components, memory devices, and applications comprised within them, work together to present an immersive multimedia environment in which a patient user may be administered TBR therapy.

As illustrated in FIG. 11, the CPU or digital signal processor 110 is coupled to the memory 112 via a bus 211. The CPU 110, as noted above, may be a multiple-core processor having N core processors. That is, the CPU 110 includes a first core 222, a second core 224, and an Nth core 230. As is known to one of ordinary skill in the art, each of the first core 222, the second core 224 and the Nth core 230 are available for supporting a dedicated application or program. Alternatively, one or more applications or programs can be distributed for processing across two or more of the available cores.

The CPU 110 may receive commands from the learning module 40, TBR Therapy Selection and Session module 50, the monitoring module 115 and/or TBR module(s) 101 that may comprise software and/or hardware. If embodied as software, the module(s) comprise instructions that are executed by the CPU 110 that issues commands to other application programs being executed by the CPU 110 and other processors.

The first core 222, the second core 224 through to the Nth core 230 of the CPU 110 may be integrated on a single integrated circuit die, or they may be integrated or coupled on separate dies in a multiple-circuit package. Designers may couple the first core 222, the second core 224 through to the Nth core 230 via one or more shared caches and they may implement message or instruction passing via network topologies such as bus, ring, mesh and crossbar topologies.

Bus 211 may include multiple communication paths via one or more wired or wireless connections, as is known in the art. The bus 211 may have additional elements, which are omitted for simplicity, such as controllers, buffers (caches), drivers, repeaters, and receivers, to enable communications. Further, the bus 211 may include address, control, and/or data connections to enable appropriate communications among the aforementioned components.

When the logic used by the TBR platform 100 is implemented in software, as is shown in FIG. 11, it should be noted that one or more of startup logic 250, management logic 260, TBR Therapy Interface logic 270, applications in application store 280 and portions of the file system 290 may be stored on any computer-readable medium (or device) for use by, or in connection with, any computer-related system or method.

In the context of this document, a computer-readable medium is an electronic, magnetic, optical, or other physical device or means that can contain or store a computer program and data for use by or in connection with a computer-related system or method. A computer-readable medium may be “local” to a component of a TBR platform 100 or may be “in the cloud,” as would be understood by one of ordinary skill in the art. The various logic elements and data stores may be embodied in any computer-readable medium for use by or in connection with an instruction execution system, apparatus, or device, such as a computer-based system, processor-containing system, or other system that can fetch the instructions from the instruction execution system, apparatus, or device and execute the instructions. In the context of this document, a “computer-readable medium” can be any means that can store, communicate, propagate, or transport the program for use by or in connection with the instruction execution system, apparatus, or device.

The computer-readable medium can be, for example but not limited to, an electronic, magnetic, optical, electromagnetic, infrared, or semiconductor system, apparatus, device, or propagation medium. More specific examples (a non-exhaustive list) of the computer-readable medium would include the following: an electrical connection (electronic) having one or more wires, a portable computer diskette (magnetic), a random-access memory (RAM) (electronic), a double data rate memory (DDR) (electronic), a read-only memory (ROM) (electronic), an erasable programmable read-only memory (EPROM, EEPROM, or Flash memory) (electronic), an optical fiber (optical), and a portable compact disc read-only memory (CDROM) (optical).

In an alternative embodiment, where one or more of the startup logic 250, management logic 260 and perhaps the TBR therapy interface interface logic 270 are implemented in hardware, the various logic may be implemented with any or a combination of the following technologies, which are each well known in the art: a discrete logic circuit(s) having logic gates for implementing logic functions upon data signals, an application specific integrated circuit (ASIC) having appropriate combinational logic gates, a programmable gate array(s) (PGA), a field programmable gate array (FPGA), etc.

The memory 112 is a non-volatile data storage device such as a flash memory or a solid-state memory device. Although depicted as a single device, the memory 112 may be a distributed memory device with separate data stores coupled to the digital signal processor 110 (or additional processor cores).

The startup logic 250 includes one or more executable instructions for selectively identifying, loading, and executing a select program for administering TBR therapies in an immersive multimedia environment. The startup logic 250 may identify, load and execute a select program based on progress data of a patient user from a previous TBR session. An exemplary select program can be found in the program store 296 of the embedded file system 290 and is defined by a specific combination of a TBR therapy algorithm 297 and user profile data 298. The exemplary select program, when executed by one or more of the core processors in the CPU 110 may operate in accordance with one or more signals provided by the monitoring module 115 in combination with control signals provided by the device(s) 158 and sensor(s) 157, 159 of an XR subsystem 102 to administer a TBR therapy to a patient user engaged in an XR environment.

The management logic 260 includes one or more executable instructions for terminating an active TBR session and/or exercise program, as well as selectively identifying, loading, and executing a more suitable replacement program. The management logic 260 is arranged to perform these functions at run time or while the TBR platform is powered and in use by an operator of an XR subsystem 102. A replacement program can be found in the program store 296 of the embedded file system 290 and, in some embodiments, may be defined by a specific combination of a TBR therapy algorithm 297 and user profile data 298.

The interface logic 270 includes one or more executable instructions for presenting, managing and interacting with external inputs to observe, configure, or otherwise update information stored in the embedded file system 290. In one embodiment, the interface logic 270 may operate in conjunction with administrator inputs loaded into the TBR module 101 and/or XR subsystem(s) 102. These inputs may include one or more programs to be deleted from or added to the program store 296. Alternatively, the inputs may include edits or changes to one or more of the programs in the program store 296. Moreover, the inputs may identify one or more changes to, or entire replacements of one or both of the startup logic 250 and the management logic 260.

The interface logic 270 enables an administrator to controllably configure and adjust a patient user's options when using a TBR platform 100. When the memory 112 is a flash memory, one or more of the startup logic 250, the management logic 260, the interface logic 270, the application programs in the application store 280 or information in the embedded file system 290 can be edited, replaced, or otherwise modified. In some embodiments, the interface logic 270 may permit a user or operator of the TBR platform 100 to search, locate, modify or replace the startup logic 250, the management logic 260, applications in the application store 280 and information in the embedded file system 290. The operator may use the resulting interface to make changes that will be implemented upon the next startup of the platform 100. Alternatively, the operator may use the resulting interface to make changes that are implemented during run time.

The embedded file system 290 includes a hierarchically arranged TBR therapy store 292. In this regard, the file system 290 may include a reserved section of its total file system capacity for the storage of information for the configuration and management of the various profile data 298 and TBR therapy algorithms 297 used by the platform 100. As shown in FIG. 11, the store 292 includes a session store 294, which includes a program store 296, which includes one or more programs for delivering TBR therapy in an immersive multimedia environment.

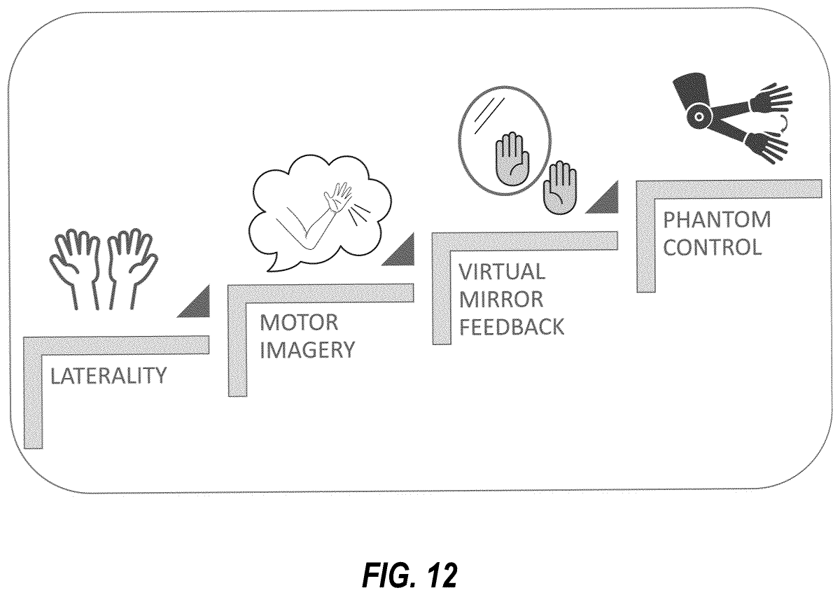

Turning now to the illustrations of FIGS. 12 through 19B, certain aspects and functionalities of exemplary embodiments of the solution previously described or mentioned or implied in this description will be illustrated in more detail.

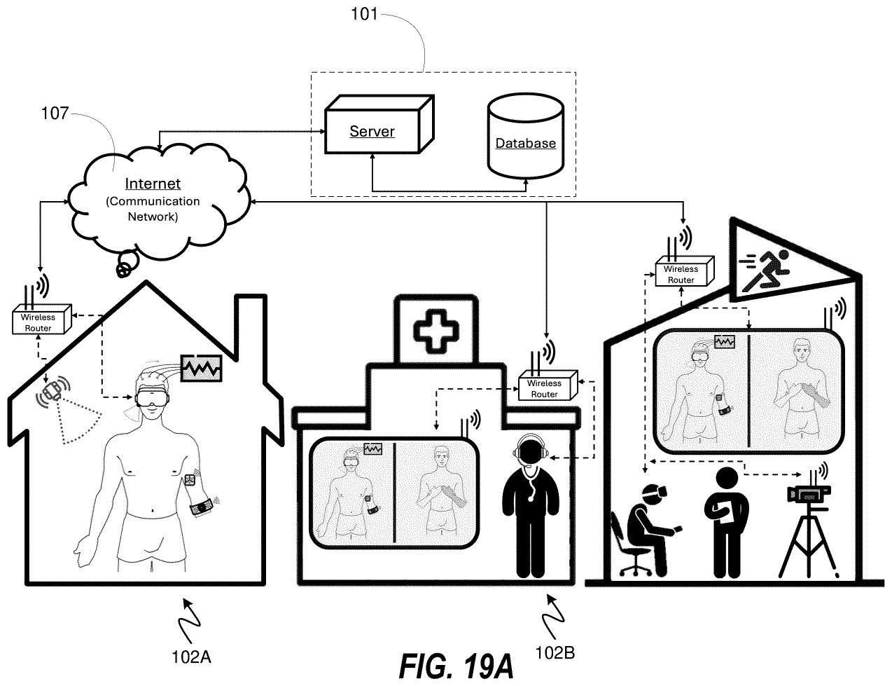

FIG. 12 is a diagram of the stepwise progression of the four primary phases of a TBR therapy plan that may be implemented in a virtual environment by and through embodiments of the solution. As can be understood from the FIG. 12 illustration, a user of the platform 100 may progress through different phases of therapy using an avatar in a virtual environment generated by the platform 100. As described in more detail at various places in this disclosure, a user may begin with laterality exercises that work to retrain the brain to distinguish right from left. As a part of laterality exercises, some embodiments may employ random hand pose generators and/or selectors, as will be described in more detail below relative to the FIG. 18A and 18B illustrations. The therapy plan may progress to a motor imagery phase before advancing to a virtual mirror feedback phase. Some embodiments may further include a phantom control phase wherein the user is trained in a virtual environment to use a real-world prosthetic.

Notably, a user of a TBR platform 100 may progress through the phases of a TBR therapy plan in any order dictated by the plan, and not necessarily in the order presented in the FIG. 12 illustration. Specifically, the order of the phases and parts of phases can be guided by external plans/decisions (i.e. directed by a therapist) or by internal decision matrixes such as scores, progress, virtual therapists guided by complex neural networks, etc. Depending on the user and the particular therapeutic needs, a TBR therapy plan may even skip some phases and incorporate others not necessarily shown and described in the FIG. 12 illustration.

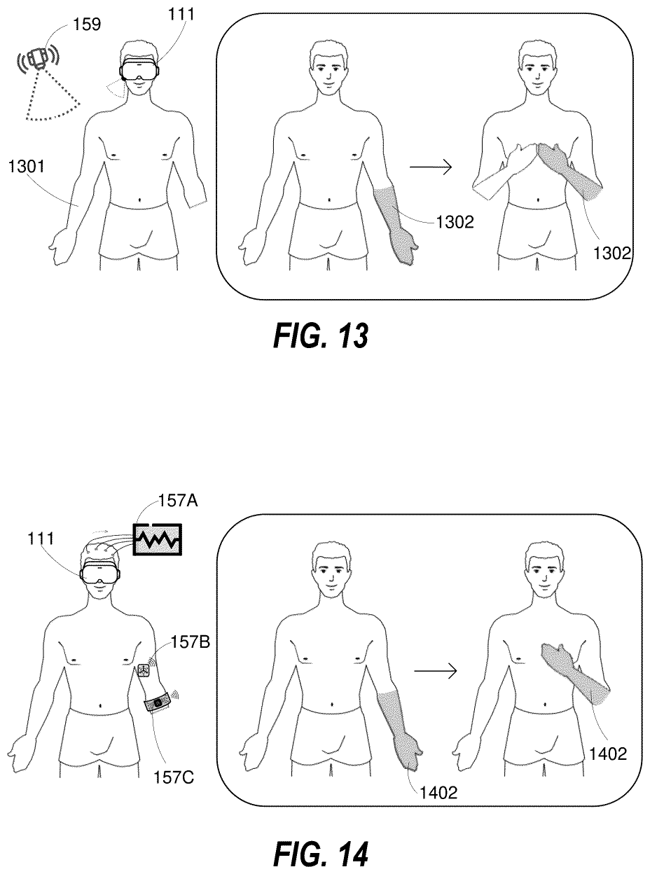

FIG. 13 illustrates an amputee user of an embodiment of the solution that leverages an XR headset 111 along with both embedded and external sensors 159 to scan the user's intact limb and mirror a copy onto the user's avatar in the XR environment to represent the user's amputated limb. As previously described, the sensors 159 could include, but are not limited to including, LiDAR, cameras, infrared cameras, accelerometers, gyroscopes, tracking gloves or other accessories, etc. These would allow the accurate scanning and mirroring of the intact limb(s) including size, position, surface texture/color, etc.

As can be understood from the illustration, the avatar, as seen by the user in the virtual environment, is illustrated in the rectangular field. The mirrored copy representing the amputated limb is shown in a “grayed out” form. As the sensors 159 recognize changes in the position and movement of the user's intact limb 1301, the platform 100 mirrors the changes with the presentation of the phantom limb 1302. In this way, the system 100 enables the user to perceive use of a limb in place of the amputated limb.

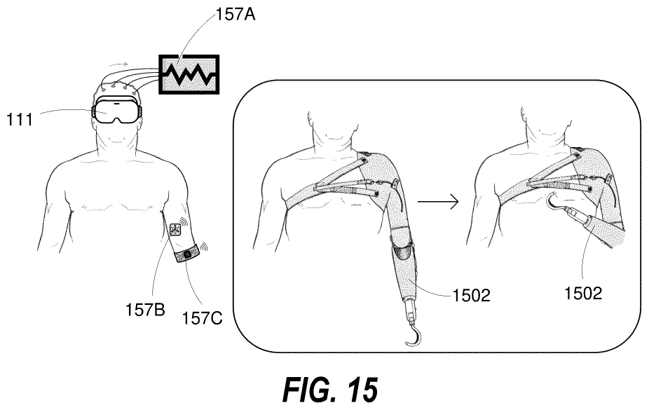

FIG. 14 illustrates an amputee user of an embodiment of the solution that leverages an XR headset 111 along with both internal and external sensors (such as a myoelectric band 157C, accelerometers/gyroscopes 157B, and EEG activity sensors 157A) to map the desired location and movement of the user's amputated/phantom limb and project an anatomically appropriate copy 1402 onto the user's avatar in the XR environment to represent the user's amputated limb. As can be further understood from the FIG. 14 illustration, the figure also depicts the amputee user controlling this phantom/avatar limb 1402 in the XR environment via the sensors 157 (such as a myoelectric band, accelerometer/gyroscope, and/or EEG, etc.).

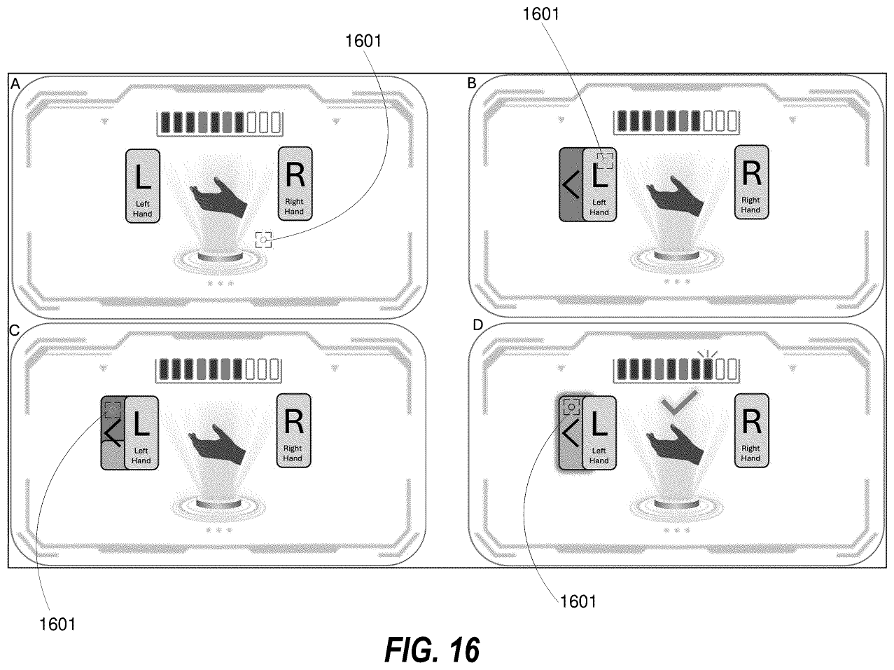

FIG. 15 illustrates an amputee user of an embodiment of the solution that leverages an XR headset 111 along with both internal and external sensors 157 (such as a myoelectric band 157C, accelerometers/gyroscopes 157B, and EEG activity sensors 157A) to map the desired location and movement of the user's amputated/phantom limb and project a virtual representation of a correctly positioned given/selected prosthetic 1502 onto the user's avatar in the XR environment. Advantageously, embodiments of the solution for a TBR platform 100 may store specifications for any number of different real-world prosthetics such that the system may project any one of them onto a user's avatar. In this way, the system 100 may offer training to a user that is customized to the specific prosthetic that the user is/will be employing in reality. For example, in the FIG. 15 illustration, a body powered prosthetic is shown, but any prosthetic with any number of degrees of freedom could be used including a myoelectric prosthetic.

Similar to that illustrated above in FIG. 14, the location of the avatar prosthesis 1502 in the XR environment is directed by user input via the sensors 157, which could come in the form of physiologic and/or neurologic sensors. These sensors can transform required body-powered movements, such as stump or scapular motion, or myoelectric activity into their virtual counterparts. This encapsulates switching motions which change prosthetic output, thus allowing one user-directed motion to control multiple prosthetic functions in the virtual environment.

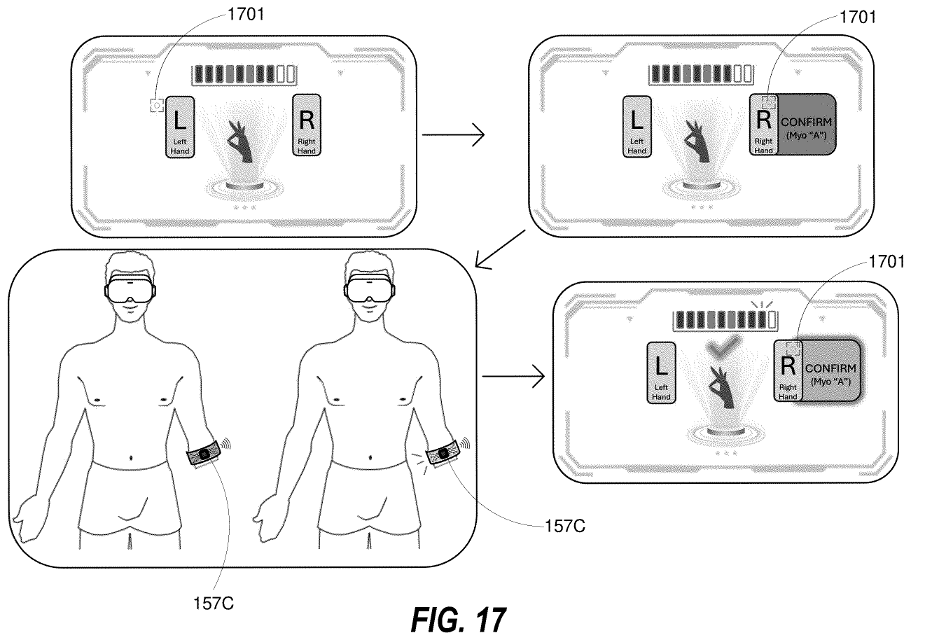

FIG. 16 illustrates an exemplary embodiment of a Gaze Directed Interaction (GDI) functionality that may be included in the user interface of certain embodiments of the solution. As previously described, the center of a user's gaze may be tracked with physiological sensors 157 embedded in the headset 111. The location of the user's gaze may be represented in the virtual environment by a reticle feature or center of gaze icon (CoGI) 1601. When the CoGI 1601 moves over a desired and selectable object in the virtual environment, such as a button or pull down menu, that object is interacted with.

To prevent inadvertent interactions, it is envisioned that embodiments of the solution may use a “double-click” functionality whereby the user's initial action with the CoGI 1601 is confirmed by a follow up action. For example, referring to the FIG. 16 illustration, a selection menu in a laterality exercise is shown progressing through four frames (A through D). The user may be an amputee user physically incapable of pushing a physical button or otherwise employing user input devices 158 that require physical actuation. Using the CoGI 1601 in the A-frame, the system 100 tracks the user's gaze to move the CoGI 1601 to the “L” button as seen in the B-frame. Directing the CoGI 1601 onto the “L” button may be recognized by the system 100 to be a selection of the “L” button, thus presenting to the left of the “L” button an arrow pane as a secondary target for the user. The secondary target may be configured to, if selected, confirm the user's intention to actuate/select the “L” button. With the arrow pane presented, the user may use his gaze to direct the CoGI 1601 over the arrow pane, as shown in the C-frame, and hold the gaze there for a predetermined amount of time. If the gaze is held onto the secondary target for the predetermined amount of time (illustrated in the C-frame as a temporal change in color of the arrow pane), the system 100 may conclude that the user intends to select the target and so actuate it, as shown in the D-frame. Advantageously, the follow up action to the initial selection is performed by moving the CoGI to a secondary target and hovering over that target for a predetermined period of time. In this way, the system 100 may provide means for quadriplegic and other amputee users to interact and engage with a TBR therapy plan in the virtual environment.

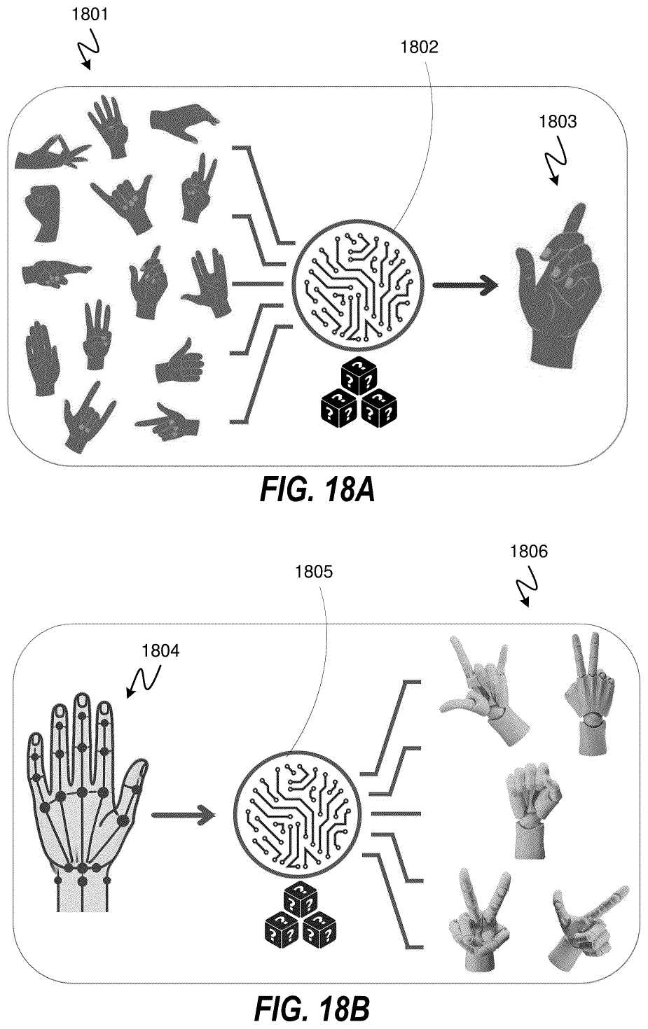

FIG. 17 is a further illustration of the GDI functionality shown and described by FIG. 16. In the FIG. 17 illustration, however, the “double-click” feature is initiated with the CoGI 1701 and completed by an external action. In this illustration, the external action is completed by activating the myoelectric band 157C. This could be done manually with an intact limb (and visualized in the XR environment in real-time) through direct interaction (i.e. pressing a button, etc.) on the myoelectric band (or another external controller 158). It could also be completed through a user's myoelectric or neural input signal 157A. As a nonlimiting example, it is envisioned that the user could assign a function (such as “pressing A”) to a particular myoelectric stimulus (such as “extending the thumb”) and then performing this action would satisfy the “double-click” and complete the GDI.

FIG. 18A illustrates an exemplary embodiment of a Random Pose Generator (RPG) functionality that may be included in certain embodiments of the solution, the exemplary embodiment configured to present a hand pose 1803 selected from a predefined set of hand poses 1801. The RPG module 1802 may be employed, for example, by a TBR therapy plan engaged in laterality exercises. The RPG module 1802 selects, perhaps using a random selection algorithm, a given hand pose 1803 from a predefined set 1801 of hand poses. The selected hand pose 1803 may be presented to a user of the TBR system 100 engaged in a virtual environment. Depending on embodiment, the RPG selector module may leverage an algorithm and/or deep learning neural network to learn which poses the user has difficulty with and present poses in increasing difficulty intervals. Furthermore, it is envisioned that this RPG could be used in training patients to use their phantom limb and/or prosthetic using certain myoelectric or other sensors. Specifically, the TBR system may require the user to “respond” to the RPG presentation by manipulating their intact or phantom limb(s) to match the laterality and pose generated by the RPG.

FIG. 18B illustrates an exemplary embodiment of a Random Pose Generator (RPG) functionality that may be included in certain embodiments of the solution, the exemplary embodiment configured to present a randomly generated hand pose 1806. The RPG module 1805 may be employed, for example, by a TBR therapy plan engaged in laterality exercises. The RPG module 1805 randomly generates a given hand pose 1806 based on predefined rules and mathematical limits that prevent generation of an unrealistic, or physiologically impossible, poses and/or digit collisions. The number and types of hand poses possible for generation by the RPG module 1805 may be dictated by the baseline number of joints and degrees of freedom provided by the virtual hand 1804 used for the poses. The generated hand pose 1806 may be presented to a user of the TBR system 100 engaged in a virtual environment.

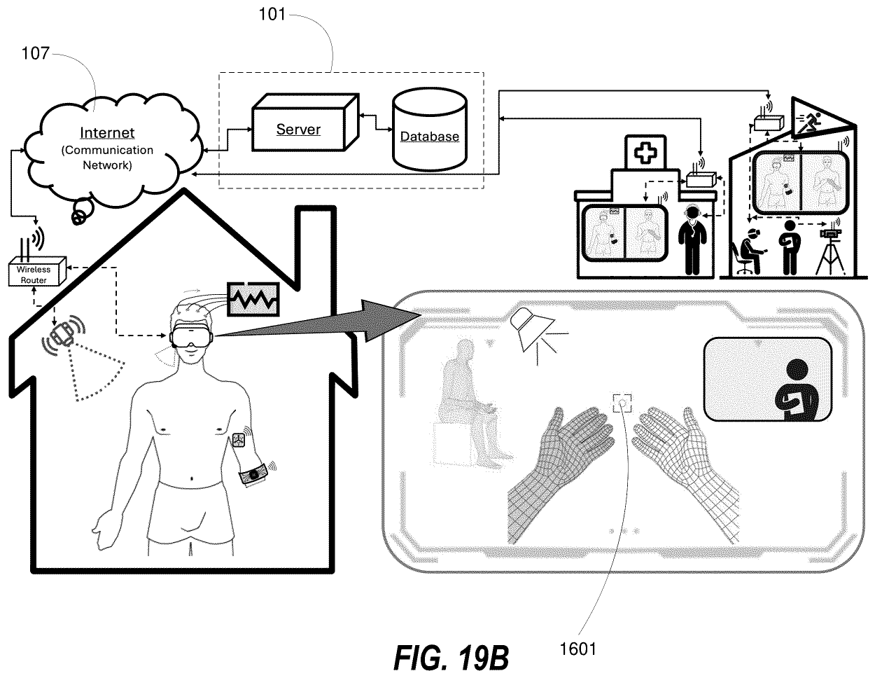

FIGS. 19A and 19B are more detailed high-level illustrations of the exemplary architecture for a TBR system 100 shown in FIGS. 1 and 4. The FIG. 19 illustrations further highlight the advantageous aspect of the solution that allows for administering TBR therapies to patients who are geographically remote from their clinician and/or family caregivers. As previously described, the patient user with XR subsystem 102A may be physically remote from other users of the system 100, such as a clinician user with subsystem 102B. Advantageously, the clinician user may interact with the patient by operating within the virtual environment himself (such as depicted in the “hospital” shown in the illustration) or by monitoring video input and the clinician “calling in” (such as depicted in the “clinic” shown in the illustration).

The illustration of FIG. 19B depicts an exemplary patient view of the virtual environment. Other users of the system 100 may experience similar views of the virtual environment, as they interact with the patient's avatar and help or guide the TBR therapy. As can be seen in the exemplary patient view a CoGI icon 1601 is presented along with virtual representations of the patient user's hands (one or both may be virtual representations of amputated limbs and/or real-life, residual limbs, as previously described). Avatars representing other user's may also be perceived. Certain embodiments may also have a “picture-in-picture” functionality so that the patient user can actually see a live video feed of another user such as a clinician user who is not necessarily engaging with the patient user via an avatar.

Certain steps in the processes or process flows described in this specification naturally precede others for the invention to function as described. However, the invention is not limited to the order of the steps described if such order or sequence does not alter the functionality of the invention. That is, it is recognized that some steps may performed before, after, or parallel (substantially simultaneously with) other steps without departing from the scope and spirit of the invention. In some instances, certain steps may be omitted or not performed without departing from the invention. Further, words such as “thereafter,” “then,” “next,” “proceed,” etc. are not intended to limit the order of the steps. These words are simply used to guide the reader through the description of the exemplary method.

Additionally, one of ordinary skill in programming is able to write computer code or identify appropriate hardware and/or circuits to implement the disclosed invention without difficulty based on the flow charts and associated description in this specification, for example. Therefore, disclosure of a particular set of program code instructions or detailed hardware devices is not considered necessary for an adequate understanding of how to make and use the invention. The inventive functionality of the claimed computer implemented processes is explained in more detail in the above description and in conjunction with the drawings, which may illustrate various process flows.

Therefore, although selected aspects have been illustrated and described in detail, it will be understood that various substitutions and alterations may be made therein without departing from the spirit and scope of the present invention, as defined by the following claims.

What is claimed is:

1. A method for administering targeted brain rehabilitation therapies in an immersive multimedia environment, the method comprising:
generating an immersive multimedia environment;
rendering a first avatar within the immersive multimedia environment, wherein the first avatar is associated with a patient user of an XR subsystem and includes a virtual representation of a lost limb of the patient user; and
administering a targeted brain rehabilitative therapy exercise to the patient user via the first avatar, wherein the therapy exercise in the immersive multimedia environment works to address somatosensory and kinesthetic symptoms associated with the patient user's lost limb.

2. The method of claim 1, further comprising:
monitoring myoelectrical signals associated with a remaining musculature of the patient user;
identifying myoelectrical signal patterns associated with stimuli presented to the patient user via the immersive multimedia environment; and
manipulating the virtual representation of the lost limb in the immersive multimedia environment.

3. The method of claim 1, wherein the targeted brain rehabilitative therapy exercise is directed to laterality recognition.

4. The method of claim 1, wherein the targeted brain rehabilitative therapy exercise is directed to imagined movements of the lost limb.

5. The method of claim 1, wherein the first avatar further includes a virtual representation of remaining, intact limb of the patient user and the targeted brain rehabilitative therapy exercise is directed to virtual mirror therapy such that the virtual representation of the lost limb is manipulated to mirror movement and positioning of the virtual representation of the patient user's remaining, intact limb.

6. A computer system for administering targeted brain rehabilitation therapies in an immersive multimedia environment, the system comprising:
a TBR module and one or more user XR subsystems collectively configured to:
generate an immersive multimedia environment;
render a first avatar within the immersive multimedia environment, wherein the first avatar is associated with a patient user of one of the one or more XR subsystems and includes a virtual representation of a lost limb of the patient user; and
administer a targeted brain rehabilitative therapy exercise to the patient user via the first avatar, wherein the therapy exercise in the immersive multimedia environment works to address somatosensory and kinesthetic symptoms associated with the patient user's lost limb.

7. The computer system of claim 6, wherein the TBR module and one or more user XR subsystems are further collectively configured to:
monitor myoelectrical signals associated with a remaining musculature of the patient user;
identify myoelectrical signal patterns associated with stimuli presented to the patient user via the immersive multimedia environment; and
manipulate the virtual representation of the lost limb in the immersive multimedia environment.

8. The computer system of claim 6, wherein the targeted brain rehabilitative therapy exercise is directed to laterality recognition.

9. The computer system of claim 6, wherein the targeted brain rehabilitative therapy exercise is directed to imagined movements of the lost limb.

10. The computer system of claim 6, wherein the first avatar further includes a virtual representation of remaining, intact limb of the patient user and the targeted brain rehabilitative therapy exercise is directed to virtual mirror therapy such that the virtual representation of the lost limb is manipulated to mirror movement and positioning of the virtual representation of the patient user's remaining, intact limb.

11. A computer system for administering targeted brain rehabilitation therapies in an immersive multimedia environment, the system comprising:
means for generating an immersive multimedia environment;
means for rendering a first avatar within the immersive multimedia environment, wherein the first avatar is associated with a patient user of an XR subsystem and includes a virtual representation of a lost limb of the patient user; and
means for administering a targeted brain rehabilitative therapy exercise to the patient user via the first avatar, wherein the therapy exercise in the immersive multimedia environment works to address somatosensory and kinesthetic symptoms associated with the patient user's lost limb.

12. The computer system of claim 11, further comprising:
means for monitoring myoelectrical signals associated with a remaining musculature of the patient user;
means for identifying myoelectrical signal patterns associated with stimuli presented to the patient user via the immersive multimedia environment; and
means for manipulating the virtual representation of the lost limb in the immersive multimedia environment.

13. The computer system of claim 11, wherein the targeted brain rehabilitative therapy exercise is directed to laterality recognition.

14. The computer system of claim 11, wherein the targeted brain rehabilitative therapy exercise is directed to imagined movements of the lost limb.

15. The computer system of claim 11, wherein the first avatar further includes a virtual representation of remaining, intact limb of the patient user and the targeted brain rehabilitative therapy exercise is directed to virtual mirror therapy such that the virtual representation of the lost limb is manipulated to mirror movement and positioning of the virtual representation of the patient user's remaining, intact limb.

16. A computer program product comprising a computer usable device having a computer readable program code embodied therein, said computer readable program code adapted to be executed to implement a method for administering targeted brain rehabilitation therapies in an immersive multimedia environment, said method comprising:
generating an immersive multimedia environment;
rendering a first avatar within the immersive multimedia environment, wherein the first avatar is associated with a patient user of an XR subsystem and includes a virtual representation of a lost limb of the patient user; and
administering a targeted brain rehabilitative therapy exercise to the patient user via the first avatar, wherein the therapy exercise in the immersive multimedia environment works to address somatosensory and kinesthetic symptoms associated with the patient user's lost limb.

17. The computer program product of claim 16, the method further comprising:
monitoring myoelectrical signals associated with a remaining musculature of the patient user;
identifying myoelectrical signal patterns associated with stimuli presented to the patient user via the immersive multimedia environment; and
manipulating the virtual representation of the lost limb in the immersive multimedia environment.

18. The computer program product of claim 16, wherein the targeted brain rehabilitative therapy exercise is directed to laterality recognition.

19. The computer program product of claim 16, wherein the targeted brain rehabilitative therapy exercise is directed to imagined movements of the lost limb.

20. The computer program product of claim 16, wherein the first avatar further includes a virtual representation of remaining, intact limb of the patient user and the targeted brain rehabilitative therapy exercise is directed to virtual mirror therapy such that the virtual representation of the lost limb is manipulated to mirror movement and positioning of the virtual representation of the patient user's remaining, intact limb.
